(19) United States
(12) Patent Application Publication
(10) Pub. No.: US 2022/0165383 A1
(43) Pub. Date: May 26, 2022

(54) GENERATING DYNAMIC ELECTRONIC USER NOTIFICATIONS TO FACILITATE SAFE PRESCRIPTION USE

(71) Applicant: Optum, Inc., Minnetonka, MN (US)

(72) Inventors: Rama S. Ravindranathan, Edison, NJ (US); Gregory J. Boss, Saginaw, MI (US); Jon Kevin Muse, Thompsons Station, TN (US); Paul J. Godden, London (GB)

(21) Appl. No.: 17/100,324

(22) Filed: Nov. 20, 2020

(51) Int. Cl. G16H 20/13; G06N 20/00; H04L 29/08

(52) U.S. Cl. CPC G16H 20/13; H04L 67/125; G06N 20/00

(57) ABSTRACT

Embodiments of the present disclosure provide methods, apparatus, systems, computing devices, and/or computing entities for causing a medication dispensing device to generate electronic user notifications. According to one embodiment, a method is provided that includes: receiving an attempt data object indicating an end user has communicated a medication access request for a medication; and responsive to receiving the object: receiving a predictive polypharmacy data object generated via processing an end user medication profile, an end user factor profile, and a polypharmacy profile for the medication using a polypharmacy prediction machine learning model indicating an inferred prediction about polypharmacy compatibility; determining, based at least in part on the predictive polypharmacy data object, a confirmation data object indicating a recommended response to the attempt data object; and transmitting the confirmation data object to the medical dispensing device to generate the electronic user notifications communicated to the end user.

### TECHNOLOGICAL FIELD

Embodiments of the present disclosure generally relate to systems and methods for causing a medication dispensing device to generate one or more dynamic electronic user notifications to facilitate safe prescription use.

### BACKGROUND

A need exists in the industry to address technical challenges related to monitoring and advising individuals in taking prescribed medications so that such individuals are not taking medications at the wrong times, at the wrong dosage, under circumstances that may result in adverse side effects, under circumstances that may result in medical abuse, and/or the like. It is with respect to these considerations and others that the disclosure herein is presented.

### BRIEF SUMMARY

In general, embodiments of the present disclosure provide methods, apparatus, systems, computing devices, computing entities, and/or the like for causing a medication dispensing device to generate one or more dynamic electronic user notifications. In accordance with one aspect of the disclosure, a method for causing a medication dispensing device to generate one or more dynamic electronic notifications is provided. In various embodiments, the method includes: receiving, by one or more processors of a user computing entity and originating from the medication dispensing device, an attempt data object, wherein the attempt data object indicates that an end user has communicated a medication access request for a medication to the medication dispensing device; and responsive to receiving the attempt data object: receiving, by the one or more processors and originating from a predictive data analysis server computing entity, a predictive polypharmacy data object for the end user, wherein: (i) the predictive polypharmacy data object is generated by the predictive data analysis computing entity via processing an end user medication profile for the end user, an end user factor profile for the end user, and a polypharmacy profile for the medication using a polypharmacy prediction machine learning model, and (ii) the predictive polypharmacy data object indicates an inferred prediction about polypharmacy compatibility of the polypharmacy profile and the end user factor profile; determining, by the one or more processors and based at least in part on the predictive polypharmacy data object, a confirmation data object, wherein the confirmation data object indicates a recommended response to the attempt data object; and transmitting the confirmation data object to the medical dispensing device, wherein the medical dispensing device is configured to generate the one or more dynamic electronic user notifications based at least in part on the confirmation data object and communicate the one or more dynamic electronic user notifications to the end user.

In accordance with another aspect of the present disclosure, an apparatus is provided. In various embodiments, the apparatus includes at least one processor and at least one memory including program code. The at least one memory and the program code are configured to, with the processor, cause the apparatus to at least: receive, originating from the medication dispensing device, an attempt data object, wherein the attempt data object indicates that an end user has communicated a medication access request for a medication to the medication dispensing device; and responsive to receiving the attempt data object: receive, originating from a predictive data analysis server computing entity, a predictive polypharmacy data object for the end user, wherein: (i) the predictive polypharmacy data object is generated by the predictive data analysis computing entity via processing an end user medication profile for the end user, an end user factor profile for the end user, and a polypharmacy profile for the medication using a polypharmacy prediction machine learning model, and (ii) the predictive polypharmacy data object indicates an inferred prediction about polypharmacy compatibility of the polypharmacy profile and the end user factor profile; determine, based at least in part on the predictive polypharmacy data object, a confirmation data object, wherein the confirmation data object indicates a recommended response to the attempt data object; and transmit the confirmation data object to the medical dispensing device, wherein the medical dispensing device is configured to generate the one or more dynamic electronic user notifications based at least in part on the confirmation data object and communicate the one or more dynamic electronic user notifications to the end user.

In accordance with yet another aspect of the present disclosure, a computer program product is provided. In particular embodiments, the computer program product includes a non-transitory computer storage medium having instructions stored therein. The instructions being configured to cause one or more computer processors to at least perform operations configured to: receive, originating from the medication dispensing device, an attempt data object, wherein the attempt data object indicates that an end user has communicated a medication access request for a medication to the medication dispensing device; and responsive to receiving the attempt data object: receive, originating from a predictive data analysis server computing entity, a predictive polypharmacy data object for the end user, wherein: (i) the predictive polypharmacy data object is generated by the predictive data analysis computing entity via processing an end user medication profile for the end user, an end user factor profile for the end user, and a polypharmacy profile for the medication using a polypharmacy prediction machine learning model, and (ii) the predictive polypharmacy data object indicates an inferred prediction about polypharmacy compatibility of the polypharmacy profile and the end user factor profile; determine, based at least in part on the predictive polypharmacy data object, a confirmation data object, wherein the confirmation data object indicates a recommended response to the attempt data object; and transmit the confirmation data object to the medical dispensing device, wherein the medical dispensing device is configured to generate the one or more dynamic electronic user notifications based at least in part on the confirmation data object and communicate the one or more dynamic electronic user notifications to the end user.

In particular embodiments, the one or more dynamic electronic user notifications identify to the end user whether to take a dosage of the medication by causing illumination in a first color indicating to the end user an approval for taking the medication, a second color indicating to the end user a disapproval for taking the medication, and a third color indicating to the end user an undetermined state for taking the medication. In some embodiments, the attempt data object is generated when the medication dispensing device is within a predefined distance from an end user computing entity, an apparatus, one or more computer processors, and/or the like. In other embodiments, the medication dispensing device detects a movement of the device and responsive to detecting the movement, sends the attempt data object to the user computing entity, the apparatus, the one or more computer processors, and/or the like.

In particular embodiments, the end user factor profile may include a set of relevant factors identified based at least in part on a medication factors data object. Accordingly, in some embodiments, the medication factors data object may be generated via processing an end user health profile for the end user using a medication factors machine learning model. Here, the medication factors data object may include a medication factor score for each factor of a plurality of factors and the medication factor score for a factor may indicate a probability of a relevance of the factor in generating the predictive polypharmacy data object.

In addition, in particular embodiments, the confirmation data object may be transmitted to a virtual assistant entity. Here, the virtual assistant entity may be configured to generate one or more assistant-originated electronic user notifications based at least in part on the confirmation data object and communicate the one or more assistant-originated electronic user notifications to the end user. Further, in particular embodiments, in some instances when the confirmation data object indicates a disapproval of the end user taking the medication, a locking data object may be transmitted to the medication dispensing device to cause the medication dispensing device to prevent the end user from accessing the medication. Accordingly, the medication dispensing device may be, for example, a storage unit for holding the medication, a covering for the storage unit, a lock for the storage unit, or a tag attached to the storage unit.

### BRIEF DESCRIPTION OF THE SEVERAL VIEWS OF THE DRAWING(S)

Having thus described the disclosure in general terms, reference will now be made to the accompanying drawings, which are not necessarily drawn to scale, and wherein:

FIG. 1 is a diagram of a system architecture that can be used in conjunction with various embodiments of the present disclosure;

FIG. 2 is a schematic of a computing entity that may be used in conjunction with various embodiments of the present disclosure;

FIG. 3 is a schematic of a user computing entity that may be used in conjunction with various embodiments of the present disclosure;

FIG. 4 is a process flow for configuring an user device to perform end user monitoring in accordance with various embodiments of the present disclosure;

FIG. 5 is a process flow for setting up remote monitoring of an end user in accordance with various embodiments of the present disclosure;

FIG. 6 is a process flow for establishing conditions and/or factors for a medication being taken by an end user in accordance with various embodiments of the present disclosure;

FIG. 7 is a process flow for evaluating an end user who is taking multiple medications in accordance with various embodiments of the present disclosure;

FIG. 8 is a process flow for monitoring an end user using an user device in accordance with various embodiments of the present disclosure;

FIG. 9 is a process flow for generating a confirmation for a medication access request in accordance with various embodiments of the present disclosure;

FIG. 10 is a process flow for remote monitoring of an end user in accordance with various embodiments of the present disclosure;

FIGS. 11A and 11B are a process flow for evaluating an end user who has requested access to medication in accordance with various embodiments of the present disclosure;

FIG. 12 is an example of displaying a message on an end user device in accordance with various embodiments of the present disclosure; and

FIG. 13 is another example of displaying a message on a user computing in accordance with various embodiments of the present disclosure.

### DETAILED DESCRIPTION OF SOME EXAMPLE EMBODIMENTS

Various embodiments of the present disclosure now will be described more fully hereinafter with reference to the accompanying drawings, in which some, but not all embodiments of the disclosure are shown. Indeed, the disclosure may be embodied in many different forms and should not be construed as limited to the embodiments set forth herein; rather, these embodiments are provided so that this disclosure will satisfy applicable legal requirements. The term “or” (also designated as “/”) is used herein in both the alternative and conjunctive sense, unless otherwise indicated. The terms “illustrative” and “exemplary” are used to be examples with no indication of quality level. Like numbers refer to like elements throughout.

Overview

Embodiments of the disclosure utilize a medication dispensing device that is used in conjunction with a container for holding one or more medications and one or more devices that are used to perform monitoring and advising an end user who is requesting/attempting to access the medication on whether he or she should take a dosage of the medication based at least in part on conditions placed on taking the medication and current factors that may influence what side effects the end user may experience from taking a dosage of the medication. In addition, polypharmacy concerns may be taken into consideration with respect to interactions that may occur as a result of the end user taking multiple medications and/or taking a medication after consuming a certain food product in close temporal proximity of taking the medication. For example, side effects may result from the medication interacting with another medication the end user has taken and/or a food product the end user has consumed. In particular embodiments, an inferred prediction may be generated as to the polypharmacy compatibility of the end user taking a dosage of the medication and current factors that may be related to the end user taking another medication and/or consuming a certain food product. In some embodiments, a predictive polypharmacy data object indicating the polypharmacy compatibility may be generated using one or more models such as, for example, one or more machine learning models that may include one or more rule-based operations and/or one or more operations that are each associated with a set of trained parameters.

In particular embodiments, the medication dispensing device may operate in concert with an end user device, virtual assistant device, and/or system within a cloud environment that is intelligent, adaptive, and facilitates monitoring and evaluating medication access requests initiated by an end user to gain access to the medication, and presenting confirmations to the end user in the form of audiovisual data informing the end user on whether he or she should or should not take a dosage of the medication.

The conditions may include, for example, instructions and/or restrictions set by a caregiver (e.g., a physician) who has prescribed the medication, contraindications defined by a pharmacist, manufacturer, and/or the like, additional restrictions related to the use of the medication in conjunction with non-prescription products such as over the counter drugs, vitamins, other non-clinical medications, and/or the like. The factors may describe the occurrence of events such as, for example, whether the end user has recently consumed a certain food product, whether the end user has or plans to engage in a particular activity such as driving a motor vehicle, and/or the like. In some embodiments, data associated with the conditions and factors may be identified, tracked, and/or collected in profiles for an end user so that the profiles can be used in conducting an evaluation on whether or not the end user should take a dosage of a medication upon the end user requesting to access the medication. In addition, in some embodiments, unknown effects associated with a specific medication may also be recorded in the profiles.

In various embodiments, the end user's various access requests for a medication may be tracked along with the accompanying applicable conditions and/or factors and may be analyzed to identify patterns of the end user's non-adherence and/or adherence to the conditions and/or factors necessary to properly and safely take his or her medications. In some embodiments, the analysis may be carried out using one or more models such as, for example, one or more machine learning and/or multivariate analytical models. Further, such patterns may be communicated to the end user and/or other personnel such as the end user's caregivers to reinforce adherence patterns and/or so that non-adherence patterns can be addressed and corrected.

Finally, in some embodiments, monitoring of the end user may be conducted if the end user is taking multiple medications and is considered a polypharmacy candidates to help track and distinguish medical conditions the end user may be experiencing between those that are due to true medical problems and those that are side effects due to interactions between multiple medications. Accordingly, such monitoring may help in identify and understand drug redundancy and/or over-prescribing the end user may be experiencing, as well as prevent the end user from experiencing prescription cascade.

### Definitions of Certain Terms

The term “medication dispensing device” may refer to a physical device that is configured to store medication and/or control access to medication stored in a medication storage component. For example, in particular embodiments, the medication dispensing device may be a container such as a medication dispenser or bottle in which medication in various forms can be stored. While in other embodiments, the medication dispensing device may be a component such as the lid or cap for a container used for storing medication. Still in other embodiments, the medication dispensing device may be a component that is associated with (e.g., attached to) a container used for storing medication. In some embodiments, the medication dispensing device may include one or more communication elements such as a speaker, one or more light-emitting devices, a display, and/or the like for providing one or more dynamic electronic user notifications to an end user. For example, the medication dispensing device in a particular embodiment may illuminate one or more lights in different colors to communication to an end user whether not he or she should take a particular medication upon the end user attempting to access the medication. In addition, the medication dispensing device may include one or more elements configured to communicate with other components, devices, entities, and/or the like. For example, in particular embodiments, the medication dispensing device may include radio-frequency identification (RFID) tags, iBeacons, Gimbal proximity beacons, BLE (Bluetooth Low Energy) transmitters, near field communication (NFC) transmitters, and/or the like. Accordingly, the medication dispensing device may be configured to transmit and/or receive information/data, transmissions, and/or the like of at least one of Bluetooth protocols, low energy Bluetooth protocols, NFC protocols, RFID protocols, IR protocols, Wi-Fi protocols, ZigBee protocols, Z-Wave protocols, 6LoWPAN protocols, and/or other short range communication protocol. Further, in particular embodiments, the medication dispensing device may include different types of sensors such as motion sensors for detecting movement of the medication dispensing device, different types of mechanisms such as a locking mechanism used in controlling access to medication, and/or the like.

The term “medication access request” may represent a detected action performed by an individual (e.g., an end user) to gain access to a medication. In various embodiments, the medication may be stored in some type of storage unit such as, for example, a container, dispenser, bottle, and/or the like. As described further herein, the end user may perform some action such as attempt to open the storage unit, move the storage unit, move within proximity of the storage unit, and/or the like, where the action in turns results in generating the medication access request.

The term “attempt data object” may refer to a data object generated based at least in part on a corresponding medication access request that describes one or more properties associated with the request that is associated with the corresponding medication access request, i.e., with the request by an individual (e.g., an end user) to gain access to a medication. In some embodiments, an attempt data object represents the end user attempting to access a container holding the medication so that the end user may take a dosage of the medication. Accordingly, the attempt data object may be communicated (e.g., transmitted) by a device associated with the container to another component, device, entity, and/or the like. For example, a medication dispensing device may detect a medication access request based at least in part on an end user attempting to gain access to medication and, as a result, may communicate the attempt data object to a computing entity such as a mobile device being used by the end user and/or a remote entity such as a server found in a cloud service. While in other embodiments, the attempt data object may be communicated based at least in part on some event such as, for example, the container (or associated device thereof) moving within a predefined distance to a computing entity. As discussed in further detail herein, the attempt data object is used in various embodiments to identify a trigger event for conducting an analysis as to whether the end user associated with the medication access request should or should not take the medication.

The term “end user health profile” may refer to a data object that represents data fields describing features pertaining to the current health state of an individual (e.g., an end user). The term “current health state” may refer to an overall picture of the end user's current health. For example, an end user health profile may identify medical conditions currently being experienced by the end user, as well as history of medical conditions experienced in the past. In addition, the end user health profile may identify current medications being taken by the end user, as well as a history of medications taken by the user. The end user health profile may provide physiological data collected on the end user such as height, weight, blood type, heart rate, blood pressure, and the like. Further, the end user health profile may provide a genetic profile for the end user and/or medical conditions the end user may be predisposed to develop, as well as family medical history. Furthermore, the end user health profile may provide recorded behavioral habits and/or psychological conditions of the end user. Accordingly, in particular embodiments, the end user health profile may be periodically updated with recent health information gathered on the end user (e.g., received from an electronic health record database server) so that the profile better reflects the end user's current health state.

The term “condition” may refer to a data object representing a requirement placed on an individual (e.g., an end user), where the requirement is placed because the individual is taking a particular medication. For example, a condition may relate to the frequency with which the end user is to take a dosage of the medication (e.g., twice a day). In another example, a condition may describe that the medication is to be taken with a meal or should not be taken with certain foods. Conditions for a medication may be based at least in part on instructions and/or restrictions defined by the a physician who has prescribed the medication. In addition, conditions for a medication may be based at least in part on contraindications defined by a pharmacist and/or manufacturer of the medication. Further, conditions for a medication may be based at least in part on known interactions resulting from taking the medication with other medications. As described further herein, in various embodiments, the conditions defined for a medication are considered at a time when the end user is requesting to access the medication so that he or she may take a dosage of the medication.

The term “factor” may refer to a data object representing an activity or event that may influence whether an individual (e.g., an end user) may experience a side effect from taking a medication, where the activity or event may in some embodiments be defined by one or more conditions related to the medication intake. For example, a condition may be placed on a medication the end user is prescribed which requires that the medication to be taken with a meal. The reason for this condition may be that the medication tends to cause an individual to suffer an upset stomach when taken without food. Therefore, in this example, a factor at a time when the end user plans (requests) to take the medication may relate to whether or not the end user has recently had a meal or is about to have a meal. Accordingly, factors may be tied to one or more conditions placed on the end user in taking the medications. In addition, in particular embodiments, one or more monitoring data objects may be monitored to identify occurrences of factors. For example, a condition placed on the end user in taking a medication may require that that the medication should not be taken when the end user has an elevated heart rate. Therefore, a factor identified for the medication may relate to whether or not the end user has recently engaged in physical activity at a time when the end user is planning (requesting) to take the medication. Accordingly, one or more monitoring data objects may be collected in identifying occurrences of this factor such as collecting the end user's heart rate through a heart rate monitor and/or collecting motion/movement data from a fitness tracker.

The term “end user medication profile” may refer to a data object that describes one or more features related to one or more medications being taken by an individual (e.g., an end user). In some embodiments, the end user medication profile may be considered a subset of the end user health profile. Accordingly, the end user medication profile may also include information on conditions imposed on an end user in taking a certain medication. For example, the medication profile may identify a condition for a particular medication indicating it should not be taken before operating a motor vehicle. As discussed further herein, the end user medication profile may be used in particular embodiments to determine whether the end user should take a dosage of a particular medication at a time when the end user has generated a medication access request.

The term “end user factor profile” may refer to a data object that describes one or more features related to one or more factors that may influence whether an individual (e.g., an end user) may take a medication during a particular time period. Such factors may include consuming certain food products, engaging in certain activities such as exercises, visiting certain locations, experiencing certain physiological conditions, and/or the like. In particular embodiments, the end user factor profile may be used in determining whether the end user should take a dosage of a particular medication at a time when the end user has generated a medication access request. For instance, the end user factor profile may indicate the end user has consumed a certain food product within a particular timeframe. Here, the consumption of the food product may be considered relevant to whether the food's consumption may have an interaction with a medication the end user is attempting to take. In some instances, the end user may have his or her genome sequenced. As a result, additional information relating to pharmacogenomics-related interactions may be identified and used in establishing factors for the end user. For example, grapefruit juice may cause an adverse interaction with certain medications taken for cancer. Therefore, an occurrence of the end user consuming grapefruit juice, as shown in the end user factor profile, may be considered relevant in determining whether the end user should or should not take a dosage of a cancer medication prescribed to the end user. As discussed further herein, factors may be associated with conditions placed on the end user's use of a medication.

The term “polypharmacy profile” may refer to a data object that represents known interactions that may occur as a result of individuals taking two or more different medications (in temporal proximity) with each other. In some embodiments, the polypharmacy profile may identify side effects that may occur as a result of an interaction between the two or more medications. Accordingly, in particular embodiments, the polypharmacy profile may be used in determining whether the end user should take a dosage of a particular medication at a time when an end user has generated a medication access request.

The term “predictive polypharmacy data object” may refer to a data object that represents an inferred prediction about polypharmacy compatibility of a polypharmacy profile for a medication and an end user factor profile for an end user who has generated a medication access request for the medication. Polypharmacy compatibility refers to the compatibility of the end user taking the medication with respect to other medications the end user is currently taking, conditions places on using the medication, as well as factors that may influence whether the user may experience side effects as a result of taking the medication. As discussed further herein, in various embodiments, an analysis may be conducted on these various conditions placed on the end user taking the medication and/or on various factors that may influence the end user's ability to take a dosage of the medication at a time when the medication access request has been generated. For example, conditions that may be placed on the end user in taking the medication may relate to a frequency at which the end user should take a dosage of the medication (e.g., twice a day), may require that the medication should be taken with meals, may require that the medication should not be taken with alcohol, may require that the end user should not operate a motor vehicle for a time period after taking the medication, and/or the like. Factors that may influence the end user's ability to take a dosage of the medication at the current time may describe whether the end user has recently taken another medication or consumed a certain food product such as grapefruit juice, whether the end user has engaged in an activity such as exercise, whether the end user plans to engage in an activity such as driving a motor vehicle, and/or the like. In some embodiments, the conditions and factors may be stored within the end user's end user medication profile and end user factor profile, respectfully.

The term “polypharmacy prediction machine learning model” may refer to a data object that describes parameters and/or hyper-parameters (e.g., defined operations) of a machine learning model that is configured to process an end user medication profile and/or an end user factor profile for an end user, along with a polypharmacy profile for a medication the end user is requesting to access, to generate a predictive polypharmacy data object. In some embodiments, the polypharmacy prediction machine learning model may include a rule-based machine learning sub-model that is configured to infer side effects the end user may likely experience based at least in part on conditions and factors found in the end user medication profile and end user factor profile, and potential interactions found in the polypharmacy profile. Accordingly, in particular embodiments, the polypharmacy prediction machine learning model may include a trained machine learning sub-model (e.g., trained using past historical end user data across one or many end users) that is configured to process the end user medication profile, end user factor profile, and polypharmacy profile to generate the predictive polypharmacy data object.

The term “confirmation data object” may refer to a data object that indicates a recommended response to an attempt data object that is determined based at least in part on a predictive polypharmacy data object. Accordingly, in particular embodiments, the confirmation data object may be used to generate one or more electronic user notifications. These electronic user notifications may comprise audiovisual data (i.e., audio data, visual data, or both audio data and visual data) for communicating to an end user whether to take a particular medication the end user has requested to access. For example, in particular embodiments, the one or more electronic user notifications may comprise visual data configured to cause illumination in a first color indicating to the end user an approval for taking the particular medication, a second color indicating to the end user a disapproval for taking the particular medication, and a third color indicating to the end user an undetermined state for taking the particular medication. While in other embodiments, the electronic user notifications may comprise audio data such as a first sound indicating to the end user an approval for taking the particular medication, a second sound indicating to the end user a disapproval for taking the particular medication, and a third sound indicating to the end user an undetermined state for taking the particular medication.

The term “locking data object” may refer to a data object that represents an instruction to a medication dispensing device to prevent an end user from accessing a particular medication. In some embodiments, the locking data object may be communicated to the medication dispensing device as a result of a confirmation data object indicating a disapproval for the end user taking the particular medication. Upon receiving the locking data object, the medication dispensing device may be configured with some type of locking mechanism to prohibit the end user from gaining access to a container in which the particular medication is stored. In some embodiments, transmitting the locking data object to the medication dispending device may cause the medication dispense device to generate and communicate to the end user one or more locking electronic user notifications, where the locking may comprise audiovisual data (i.e., audio data, visual data, or both audio data and visual data) notifying the end user that the end user is prevented from accessing a particular medication.

### Exemplary Technical Contributions

One technical contribution of various embodiments of the present disclosure relates to improving network reliability of a tripartite medication dispensing system that includes a server, an end user computing entity, and a medication dispensing device by decoupling the network connection between the end user computing entity and the server from the network connection between the end user computing entity and the medication dispensing device. For example, the network connection between the end user computing entity and the server may be a wide area network connection, while the network connection between the end user computing entity and the medication dispensing device may be a local area network connection. As another example, the network connection between the end user computing entity and the server may be a wide area network connection, while the network connection between the end user computing entity and the medication dispensing device may be a Bluetooth connection. By decoupling the network connection between the end user computing entity and the server from the network connection between the end user computing entity and the medication dispensing device, various embodiments of the present invention ensure that network failures with respect to one of the two networks does not affect the overall operability of the tripartite medication dispensing system, thus in turn improving the network reliability of the tripartite medication dispensing system. Moreover, in some embodiments, decoupling the network connection between the end user computing entity and the server from the network connection between the end user computing entity and the medication dispensing device can enhance the network transmission efficiency of the tripartite medication dispensing system as each network connection can be adapted to more efficiently serve the connection needs of the two parties involved rather than the more demand connection needs of a tripartite connection arrangement.

Individuals can be prescribed various medications that are customized for them based at least in part on their health and interaction with existing prescriptions. This can be communicated by an attending physician and/or a pharmacist. However, an individual may have difficulty taking medications at proper intervals, particularly because some physicians' instructions can be confusing or imprecise. In addition, an individual may have difficulty keeping track of the number of conditions placed on taking certain medications and other factors that may influence whether the individual experiences side effects. What is particularly confusing can be the interactions that can occur between medications prescribed at different times by the same physician or by a different physician (including time restrictions). This can be especially true for older individuals who may be prescribed multiple medications that are taken on a daily basis, some of which live on their own and are solely responsible for administering their own medications. Such individuals oftentimes need assistance in knowing the right time to take medications and the circumstances (e.g., conditions and/or factors) in which taking a medication may lead to adverse side effects. Finally, some individuals may be subject to medical abuse (e.g. addicts, family, or friends having access to medications) and may need additional assistance.

Accordingly, various embodiments of the present disclosure disclose a solution that is configured to effectively address the concerns many individuals experience with taking medications at the wrong time, taking too much of a particular medication, causing interactions that can lead to side effects as a result of taking a combination of medications and/or taking a medication after consuming a certain food product, and/or being victims of medically abusive actions. Specifically, various embodiments of the disclosure provide electronic confirmation notifications in the form of audiovisual data that identify to an end user whether he or she should or should not take a dosage of a medication upon the end user requesting access to the medication. In particular embodiments, such electronic confirmation notifications are provided to the end user using a device that is or is associated with a medication storage unit used for storing the medication. Accordingly, the device may be configured to provide one or more electronic user notifications that can be physically detected (e.g., seen and/or heard) by the end user to communicate to the end user a clear indication as to whether or not he or she should take a dosage of the medication.

In addition, various embodiments of the present disclosure are configured to identify, track, and/or store conditions and factors that may be relevant with respect to the end user taking a dosage of a medication at a given time. These embodiments may use such conditions and factors in generating confirmation notifications for the end user. Further, various embodiments are configured to consider individuals who are taking multiple medications (e.g., polypharmacy candidates) and interactions that may occur from taking one or more of the medications in close proximity with respect to time. Accordingly, these embodiments that may generate confirmation notifications for the end user that describe whether he or she should take a dosage of a medication when the end user requests access to the medication, where the noted medication intake recommendation is determined based at least in part on consideration of various conditions, factors, and/or interactions. As a result, embodiments of the disclosure can relieve the burden of the end user of having to self-administer medications that can lead to taking medications at the wrong time, taking too much of a particular medication, and/or taking a medication at a time that can lead to adverse side effects due to interactions.

Finally, various embodiments of the disclosure can address technical issues that may arise when an end user loses network connectivity such as Internet service between an end user device and an application server. In some embodiments, the disclosed solution may be operable in isolation without the need to communicate with a remote system and/or service to provide the end user with confirmation notifications when the end user is requesting to access a medication. For instance, various embodiments may involve a device of the end user (e.g., a mobile device) that works in concert with a medication dispensing device over a separate communication channel, including a short-range communication channel such as a Bluetooth channel. Here, the end user device may store the various conditions, factors, and/or interactions so that the device can conduct an evaluation and provide a confirmation notification as to whether the end user should take a dosage of the medication. Accordingly, the end user device can transmit the confirmation notification over the communication channel to the medication dispensing device so that the medication dispensing device can provide one or more electronic user notifications to the end user. Such capabilities can be very useful (and in some instances critical) when the end user temporally loses network connectivity over a time period in which the end user is still required to take medications. In some embodiments, the medication dispensing device may be configured so that it can conduct the evaluation on its own without having to communicate with application servers. Thus, various embodiments of the present disclosure make major technical contributions to improving the network reliability of monitoring and administering of medications to end users.

### Computer Program Products, Systems, Methods, and Computing Entities

Embodiments of the present disclosure may be implemented in various ways, including as computer program products that comprise articles of manufacture. Such computer program products may include one or more software components including, for example, software objects, methods, data structures, and/or the like. A software component may be coded in any of a variety of programming languages. An illustrative programming language may be a lower-level programming language such as an assembly language associated with a particular hardware architecture and/or operating system platform. A software component comprising assembly language instructions may require conversion into executable machine code by an assembler prior to execution by the hardware architecture and/or platform. Another example programming language may be a higher-level programming language that may be portable across multiple architectures. A software component comprising higher-level programming language instructions may require conversion to an intermediate representation by an interpreter or a compiler prior to execution.

Other examples of programming languages include, but are not limited to, a macro language, a shell or command language, a job control language, a script language, a database query or search language, and/or a report writing language. In one or more example embodiments, a software component comprising instructions in one of the foregoing examples of programming languages may be executed directly by an operating system or other software component without having to be first transformed into another form. A software component may be stored as a file or other data storage construct. Software components of a similar type or functionally related may be stored together such as, for example, in a particular directory, folder, or library. Software components may be static (e.g., pre-established or fixed) or dynamic (e.g., created or modified at the time of execution).

A computer program product may include a non-transitory computer-readable storage medium storing applications, programs, program modules, scripts, source code, program code, object code, byte code, compiled code, interpreted code, machine code, executable instructions, and/or the like (also referred to herein as executable instructions, instructions for execution, computer program products, program code, and/or similar terms used herein interchangeably). Such non-transitory computer-readable storage media include all computer-readable media (including volatile and non-volatile media).

In one embodiment, a non-volatile computer-readable storage medium may include a floppy disk, flexible disk, hard disk, solid-state storage (SSS) (e.g., a solid state drive (SSD), solid state card (SSC), solid state module (SSM), enterprise flash drive, magnetic tape, or any other non-transitory magnetic medium, and/or the like. A non-volatile computer-readable storage medium may also include a punch card, paper tape, optical mark sheet (or any other physical medium with patterns of holes or other optically recognizable indicia), compact disc read only memory (CD-ROM), compact disc-rewritable (CD-RW), digital versatile disc (DVD), Blu-ray disc (BD), any other non-transitory optical medium, and/or the like. Such a non-volatile computer-readable storage medium may also include read-only memory (ROM), programmable read-only memory (PROM), erasable programmable read-only memory (EPROM), electrically erasable programmable read-only memory (EEPROM), flash memory (e.g., Serial, NAND, NOR, and/or the like), multimedia memory cards (MMC), secure digital (SD) memory cards, SmartMedia cards, CompactFlash (CF) cards, Memory Sticks, and/or the like. Further, a non-volatile computer-readable storage medium may also include conductive-bridging random access memory (CBRAM), phase-change random access memory (PRAM), ferroelectric random-access memory (FeRAM), non-volatile random-access memory (NVRAM), magnetoresistive random-access memory (MRAM), resistive random-access memory (RRAM), Silicon-Oxide-Nitride-Oxide-Silicon memory (SONOS), floating junction gate random access memory (FJG RAM), Millipede memory, racetrack memory, and/or the like.

In one embodiment, a volatile computer-readable storage medium may include random access memory (RAM), dynamic random access memory (DRAM), static random access memory (SRAM), fast page mode dynamic random access memory (FPM DRAM), extended data-out dynamic random access memory (EDO DRAM), synchronous dynamic random access memory (SDRAM), double data rate synchronous dynamic random access memory (DDR SDRAM), double data rate type two synchronous dynamic random access memory (DDR2 SDRAM), double data rate type three synchronous dynamic random access memory (DDR3 SDRAM), Rambus dynamic random access memory (RDRAM), Twin Transistor RAM (TTRAM), Thyristor RAM (T-RAM), Zero-capacitor (Z-RAM), Rambus in-line memory module (RIMM), dual in-line memory module (DIMM), single in-line memory module (SIMM), video random access memory (VRAM), cache memory (including various levels), flash memory, register memory, and/or the like. It will be appreciated that where embodiments are described to use a computer-readable storage medium, other types of computer-readable storage media may be substituted for or used in addition to the computer-readable storage media described above.

As should be appreciated, various embodiments of the present disclosure may also be implemented as methods, apparatus, systems, computing devices, computing entities, and/or the like. As such, embodiments of the present disclosure may take the form of a data structure, apparatus, system, computing device, computing entity, and/or the like executing instructions stored on a computer-readable storage medium to perform certain steps or operations. Thus, embodiments of the present disclosure may also take the form of an entirely hardware embodiment, an entirely computer program product embodiment, and/or an embodiment that comprises a combination of computer program products and hardware performing certain steps or operations.

Embodiments of the present disclosure are described below with reference to block diagrams and flowchart illustrations. Thus, it should be understood that each block of the block diagrams and flowchart illustrations may be implemented in the form of a computer program product, an entirely hardware embodiment, a combination of hardware and computer program products, and/or apparatus, systems, computing devices, computing entities, and/or the like carrying out instructions, operations, steps, and similar words used interchangeably (e.g., the executable instructions, instructions for execution, program code, and/or the like) on a computer-readable storage medium for execution. For example, retrieval, loading, and execution of code may be performed sequentially, such that one instruction is retrieved, loaded, and executed at a time. In some exemplary embodiments, retrieval, loading, and/or execution may be performed in parallel, such that multiple instructions are retrieved, loaded, and/or executed together. Thus, such embodiments can produce specifically configured machines performing the steps or operations specified in the block diagrams and flowchart illustrations. Accordingly, the block diagrams and flowchart illustrations support various combinations of embodiments for performing the specified instructions, operations, or steps.

### Exemplary System Architectures

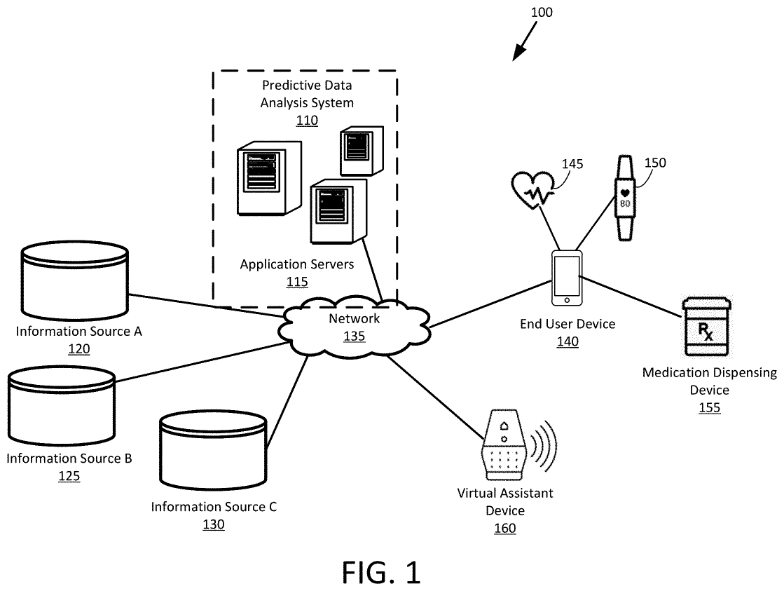

FIG. 1 provides an illustration of a system architecture 100 involving a predictive data analysis system 110 that may be used in accordance with various embodiments of the disclosure. The architecture 100 includes various components involved in analyzing medication access requests made by end users to access medications to obtain recommendations regarding whether the end users should or should not take the medication at the time they are making the requests. As detailed herein, the results of the analysis may then be used in communicating one or more electronic user notifications to communicate to the end users whether or not to take the medications they are attempting to access.

Here, the predictive data analysis system 110 may include one or more application servers 115 that may be in communication with various information sources 120, 125, 130 over one or more networks 135 (e.g., two separate network connections) to gather information from these sources 120, 125, 130. These various information sources 120, 125, 130 may include, for example, systems for medical caregivers, pharmacist, health insurance providers, credit card providers, banking institutions, retailers, and/or the like. In addition, the one or more application servers 115 may be in communication with an end user device 140 for an individual (e.g., an end user) such as the individual's desktop or laptop computer and/or a mobile device such as a smartphone. The end user device 140 may serve as a monitoring device that monitors the end user's requests to gain access to medications, as well as detect monitoring data objects in various embodiments. Accordingly, the application servers 115 may gather information (e.g., attempts and/or monitoring data objects) from the end user device 140, as well as communicate information to the device 140.

The end user device 140 may be in communication with other devices and/or components associated with the end user. For example, in some embodiments, the end user device 140 may be in communication with a heart rate monitor 145, a fitness tracker 150, and/or the like to gather information on physiological conditions of the end user which can be communicated (e.g., via Bluetooth) to the end user device 140. As detailed further herein, such information may be used in determining whether the end user should or should not take a medication at a time when the end user is requesting access to the medication.

In addition, the end user device 140 may be in communication (e.g., via RFID, Bluetooth, and/or the like) with a medication dispensing device 155. In various embodiments, the medication dispensing device 155 is associated with some type of storage unit (e.g., dispenser, container, bottle, and/or the like) that is used in storing medication. In some embodiments, the medication dispensing device 155 may contain the storage unit, or may be used in conjunction with an external storage unit. For example, the medication dispensing device 155 may be a lid or cap used for the storage unit or a tag attached to the storage unit.

As detailed further herein, the medication dispensing device 155 is configured in various embodiments to communicate with the end user by providing one or more dynamic electronic user notifications when the end user is requesting access to the medication stored in the storage unit to notify the end user (e.g., confirm with the user) on whether he or she should take the medication at that time. For example, the medication dispensing device 155 may include some type of lighting element that illuminates a first color (e.g., green) to confirm to the end user that he or she should take the medication, a second color (e.g., red) to warn the end user not to take the medication, and/or a third color (e.g., yellow) to indicate that a determination could not be made as to whether or not the end user should take the medication. In other instances, the medication dispensing device 155 may include a display screen and/or speaker that may be used to communicate with the end user. Furthermore, in some embodiments, the medication dispensing device 155 may include some type of locking mechanism to prevent the end user from gaining access to the medication if a notification is received that the end user should not have access to the medication at the time he or she is requesting access.

The architecture 100 may also include other components and/or devices that are used to communicate with the end user. For instance, in particular embodiments, the architecture 100 may include a virtual assistant device 160 that may also be configured to communication with the end user when the end user is requesting to gain access to the medication stored in the storage unit to confirm whether the end user should take the medication at that time. For example, the virtual assistant device 160 may be Apple Siri®, Google Home™, Amazon Echo™, and/or the like. Accordingly, the virtual assistant device 160 may be configured to communicate with the end user by providing audiovisual data to notify the end user about whether he or she should take a dosage of the medication.

The application server(s) 115 found within the predictive data analysis system 110 may be made up of several servers, storage media, layers, and/or other components, which may be chained or otherwise configured to interact and/or perform tasks as detailed herein. In addition, the application server(s) 115 may include any appropriate hardware and/or software for interacting with the information sources 120, 125, 130 and the end user device 140 as needed to execute aspects of one or more applications for conducting processes that involve gathering and analyzing information from the sources 120, 125, 130 and device 140, and handling data access and business logic for such.

As noted, the application server(s) 115, information sources 120, 125, 130 and end user device 140, and in some instances the virtual assistant device 160 and/or other components (e.g., the heart rate monitor 145, a fitness tracker 150, etc.), may communicate with one another over one or more networks 135. Depending on the embodiment, these networks 135 may comprise any type of known network, such as a land area network (LAN), wireless land area network (WLAN), wide area network (WAN), metropolitan area network (MAN), cellular network, the Internet, etc., or combination thereof. In addition, these networks 135 may comprise any combination of standard communication technologies and protocols. For example, communications may be carried over the networks 135 by link technologies such as Ethernet, 802.11, CDMA, 3G, 4G, 5G, or digital subscriber line (DSL). Further, the networks 135 may support a plurality of networking protocols, including the hypertext transfer protocol (HTTP), the transmission control protocol/internet protocol (TCP/IP), or the file transfer protocol (FTP), and the data transferred over the networks 135 may be encrypted using technologies such as, for example, transport layer security (TLS), secure sockets layer (SSL), and internet protocol security (IPsec).

In some embodiments, the end user device 140 may be configured to operate in concert with the medication dispensing device 155 and/or virtual assistant device 160 to perform various aspects of the disclosure without having to communicate with the predictive data analysis system 110. While in other embodiments, the medication dispensing device 155 and/or virtual assistant device 160 may be configured to perform various aspects of the disclosure without having to communicate with the end user device 140 and/or the predictive data analysis system 110. Those skilled in the art will recognize FIG. 1 represents but one possible configuration of the system architecture 100, and that variations are possible with respect to the protocols, facilities, components, technologies, and equipment used.

### Exemplary Computing Entity

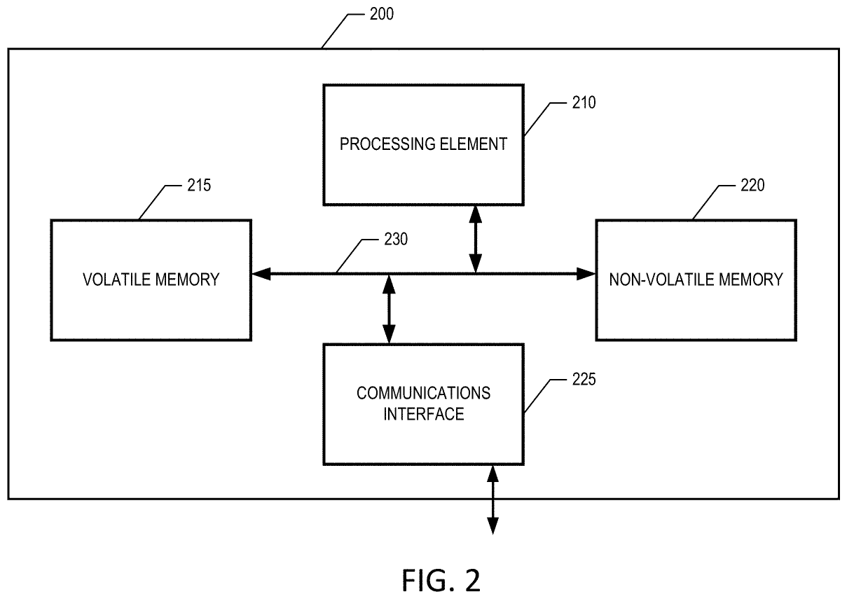

FIG. 2 provides a schematic of a computing entity 200 that may be used in accordance with various embodiments of the present disclosure. For instance, the computing entity 200 may be one or more of the application servers 115 found within the predictive data analysis system 110, and in some instances the end user device 140, previously described in FIG. 1. In general, the terms computing entity, entity, device, system, and/or similar words used herein interchangeably may refer to, for example, one or more computers, computing entities, desktop computers, mobile phones, tablets, phablets, notebooks, laptops, distributed systems, items/devices, terminals, servers or server networks, blades, gateways, switches, processing devices, processing entities, set-top boxes, relays, routers, network access points, base stations, the like, and/or any combination of devices or entities adapted to perform the functions, operations, and/or processes described herein. Such functions, operations, and/or processes may include, for example, transmitting, receiving, operating on, processing, displaying, storing, determining, creating/generating, monitoring, evaluating, comparing, and/or similar terms used herein interchangeably. In one embodiment, these functions, operations, and/or processes can be performed on data, content, information, and/or similar terms used herein interchangeably.

Although illustrated as a single computing entity, those of ordinary skill in the art should appreciate that the computing entity 200 shown in FIG. 2 may be embodied as a plurality of computing entities, tools, and/or the like operating collectively to perform one or more processes, methods, and/or steps. As just one non-limiting example, the computing entity 200 may comprise a plurality of individual data tools, each of which may perform specified tasks and/or processes.

Depending on the embodiment, the computing entity 200 may include one or more network and/or communications interfaces 225 for communicating with various computing entities, such as by communicating data, content, information, and/or similar terms used herein interchangeably that can be transmitted, received, operated on, processed, displayed, stored, and/or the like. Thus, in certain embodiments, the computing entity 200 may be configured to receive data from one or more data sources and/or devices as well as receive data indicative of input, for example, from a device.

The networks used for communicating may include, but are not limited to, any one or a combination of different types of suitable communications networks such as, for example, cable networks, public networks (e.g., the Internet), private networks (e.g., frame-relay networks), wireless networks, cellular networks, telephone networks (e.g., a public switched telephone network), or any other suitable private and/or public networks. Further, the networks may have any suitable communication range associated therewith and may include, for example, global networks (e.g., the Internet), MANs, WANs, LANs, or PANs. In addition, the networks may include any type of medium over which network traffic may be carried including, but not limited to, coaxial cable, twisted-pair wire, optical fiber, a hybrid fiber coaxial (HFC) medium, microwave terrestrial transceivers, radio frequency communication mediums, satellite communication mediums, or any combination thereof, as well as a variety of network devices and computing platforms provided by network providers or other entities.

Accordingly, such communication may be executed using a wired data transmission protocol, such as fiber distributed data interface (FDDI), digital subscriber line (DSL), Ethernet, asynchronous transfer mode (ATM), frame relay, data over cable service interface specification (DOCSIS), or any other wired transmission protocol. Similarly, the computing entity 200 may be configured to communicate via wireless external communication networks using any of a variety of protocols, such as general packet radio service (GPRS), Universal Mobile Telecommunications System (UMTS), Code Division Multiple Access 2000 (CDMA2000), CDMA2000 1× (1×RTT), Wideband Code Division Multiple Access (WCDMA), Global System for Mobile Communications (GSM), Enhanced Data rates for GSM Evolution (EDGE), Time Division-Synchronous Code Division Multiple Access (TD-SCDMA), Long Term Evolution (LTE), Evolved Universal Terrestrial Radio Access Network (E-UTRAN), Evolution-Data Optimized (EVDO), High Speed Packet Access (HSPA), High-Speed Downlink Packet Access (HSDPA), IEEE 802.11 (Wi-Fi), Wi-Fi Direct, 802.16 (WiMAX), ultra-wideband (UWB), infrared (IR) protocols, near field communication (NFC) protocols, Wibree, Bluetooth protocols, wireless universal serial bus (USB) protocols, and/or any other wireless protocol. The computing entity 200 may use such protocols and standards to communicate using Border Gateway Protocol (BGP), Dynamic Host Configuration Protocol (DHCP), Domain Name System (DNS), File Transfer Protocol (FTP), Hypertext Transfer Protocol (HTTP), HTTP over TLS/SSL/Secure, Internet Message Access Protocol (IMAP), Network Time Protocol (NTP), Simple Mail Transfer Protocol (SMTP), Telnet, Transport Layer Security (TLS), Secure Sockets Layer (SSL), Internet Protocol (IP), Transmission Control Protocol (TCP), User Datagram Protocol (UDP), Datagram Congestion Control Protocol (DCCP), Stream Control Transmission Protocol (SCTP), HyperText Markup Language (HTML), and/or the like.

In addition, in various embodiments, the computing entity 200 includes or is in communication with one or more processing elements 210 (also referred to as processors, processing circuitry, and/or similar terms used herein interchangeably) that communicate with other elements within the computing entity 200 via a bus 230, for example, or network connection. As will be understood, the processing element 210 may be embodied in several different ways. For example, the processing element 210 may be embodied as one or more complex programmable logic devices (CPLDs), microprocessors, multi-core processors, coprocessing entities, application-specific instruction-set processors (ASIPs), and/or controllers. Further, the processing element 210 may be embodied as one or more other processing devices or circuitry. The term circuitry may refer to an entirely hardware embodiment or a combination of hardware and computer program products. Thus, the processing element 210 may be embodied as integrated circuits, application specific integrated circuits (ASICs), field programmable gate arrays (FPGAs), programmable logic arrays (PLAs), hardware accelerators, other circuitry, and/or the like. As will therefore be understood, the processing element 210 may be configured for a particular use or configured to execute instructions stored in volatile or non-volatile media or otherwise accessible to the processing element 210. As such, whether configured by hardware, computer program products, or a combination thereof, the processing element 210 may be capable of performing steps or operations according to embodiments of the present disclosure when configured accordingly.

In various embodiments, the computing entity 200 may include or be in communication with non-volatile media (also referred to as non-volatile storage, memory, memory storage, memory circuitry and/or similar terms used herein interchangeably). For instance, the non-volatile storage or memory may include one or more non-volatile storage or memory media 220, such as hard disks, ROM, PROM, EPROM, EEPROM, flash memory, MMCs, SD memory cards, Memory Sticks, CBRAM, PRAM, FeRAM, RRAM, SONOS, racetrack memory, and/or the like. As will be recognized, the non-volatile storage or memory media 220 may store files, databases, database instances, database management system entities, images, data, applications, programs, program modules, scripts, source code, object code, byte code, compiled code, interpreted code, machine code, executable instructions, and/or the like. The term database, database instance, database management system entity, and/or similar terms used herein interchangeably and, in a general sense, to refer to a structured or unstructured collection of information/data that is stored in a computer-readable storage medium.

In particular embodiments, the memory media 220 may also be embodied as a data storage device or devices, as a separate database server or servers, or as a combination of data storage devices and separate database servers. Further, in some embodiments, the memory media 220 may be embodied as a distributed repository such that some of the stored information/data is stored centrally in a location within the system and other information/data is stored in one or more remote locations. Alternatively, in some embodiments, the distributed repository may be distributed over a plurality of remote storage locations only. As already discussed, various embodiments contemplated herein communicate with various information sources and/or devices in which some or all the information/data required for various embodiments of the disclosure may be stored.

In various embodiments, the computing entity 200 may further include or be in communication with volatile media (also referred to as volatile storage, memory, memory storage, memory circuitry and/or similar terms used herein interchangeably). For instance, the volatile storage or memory may also include one or more volatile storage or memory media 215 as described above, such as RAM, DRAM, SRAM, FPM DRAM, EDO DRAM, SDRAM, DDR SDRAM, DDR2 SDRAM, DDR3 SDRAM, RDRAM, RIMM, DIMM, SIMM, VRAM, cache memory, register memory, and/or the like. As will be recognized, the volatile storage or memory media 215 may be used to store at least portions of the databases, database instances, database management system entities, data, images, applications, programs, program modules, scripts, source code, object code, byte code, compiled code, interpreted code, machine code, executable instructions, and/or the like being executed by, for example, the processing element 210. Thus, the databases, database instances, database management system entities, data, images, applications, programs, program modules, scripts, source code, object code, byte code, compiled code, interpreted code, machine code, executable instructions, and/or the like may be used to control certain aspects of the operation of the computing entity 200 with the assistance of the processing element 210 and operating system.

As will be appreciated, one or more of the computing entity's components may be located remotely from other computing entity components, such as in a distributed system. Furthermore, one or more of the components may be aggregated and additional components performing functions described herein may be included in the computing entity 200. Thus, the computing entity 200 can be adapted to accommodate a variety of needs and circumstances.

### Exemplary Mobile Computing Entity

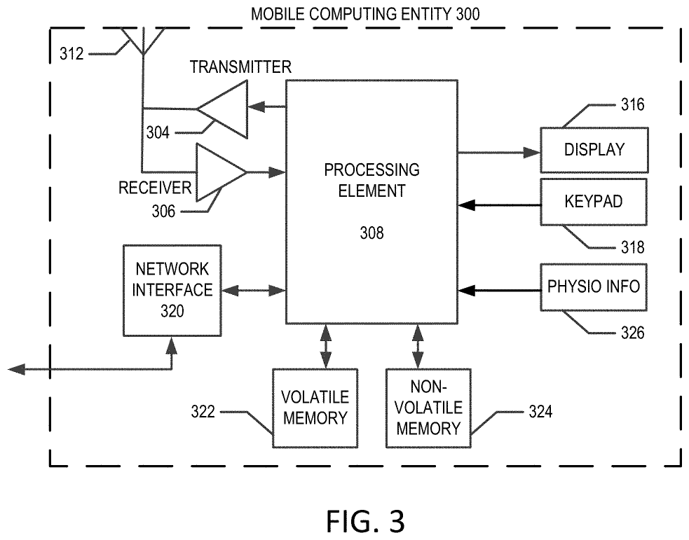

FIG. 3 provides an illustrative schematic representative of a mobile computing entity 300 that can be used in conjunction with embodiments of the present disclosure. For example, the mobile computing entity may be the end user device 140 as previously discussed in FIG. 1. Accordingly, in various embodiments, the mobile computing entity 300 may be any mobile device such as a smartphone, tablet, computing device, and/or the like that may be in communication with one or more physiological monitoring components/elements 326 (e.g., heart rate monitor 145, fitness tracker 150, etc.) that are configured to gather physiological data on an end user of the entity 300 such as, for example, heart rate, blood pressure, breathing rate, stress level, movement from activity, and the like and provide the physiological data thereof. In addition, the mobile computing entity 300 may be communication with other components and/or devices such as a virtual assistant device 160.

As shown in FIG. 3, the mobile computing entity 300 can include an antenna 312, a transmitter 304 (e.g., radio), a receiver 306 (e.g., radio), and a processing element 308 that provides signals to and receives signals from the transmitter 304 and receiver 306, respectively. The signals provided to and received from the transmitter 304 and the receiver 306, respectively, may include signaling information/data in accordance with an air interface standard of applicable wireless systems to communicate with various entities, such as the predictive data analysis system 110 and/or the like. In an example embodiment, the transmitter 304 and/or receiver 306 are configured to communicate via one or more SRC protocols. For example, the transmitter 304 and/or receiver 306 may be configured to transmit and/or receive information/data, transmissions, and/or the like of at least one of Bluetooth protocols, low energy Bluetooth protocols, NFC protocols, RFID protocols, IR protocols, Wi-Fi protocols, ZigBee protocols, Z-Wave protocols, 6LoWPAN protocols, and/or other short range communication protocol. In various embodiments, the antenna 312, transmitter 304, and receiver 306 may be configured to communicate via one or more long range protocols, such as GPRS, UMTS, CDMA200, 1×RTT, WCDMA, GSM, EDGE, TD-SCDMA, LTE, E-UTRAN, EVDO, HSPA, HSDPA, Wi-Fi, Wi-Fi Direct, WiMAX, and/or the like.

In this regard, the mobile computing entity 300 may be capable of operating with one or more air interface standards, communication protocols, modulation types, and access types. More particularly, the mobile computing entity 300 may operate in accordance with any of a number of wireless communication standards and protocols. In a particular embodiment, the mobile computing entity 300 may operate in accordance with multiple wireless communication standards and protocols, such as GPRS, UMTS, CDMA200, 1×RTT, WCDMA, TD-SCDMA, LTE, E-UTRAN, EVDO, HSPA, HSDPA, Wi-Fi, WiMAX, UWB, IR protocols, Bluetooth protocols, USB protocols, and/or any other wireless protocol.

Via these communication standards and protocols, the mobile computing entity 300 can communicate with various other devices using concepts such as Unstructured Supplementary Service information/data (USSD), Short Message Service (SMS), Multimedia Messaging Service (MMS), Dual-Tone Multi-Frequency Signaling (DTMF), and/or Subscriber Identity Module Dialer (SIM dialer). The mobile computing entity 300 can also download changes, add-ons, and updates, for instance, to its firmware, software (e.g., including executable instructions, applications, program modules), and operating system.

According to one embodiment, the mobile computing entity 300 may include location determining aspects, devices, modules, functionalities, and/or similar words used herein interchangeably. For example, the mobile computing entity 300 may include outdoor positioning aspects, such as a location module adapted to acquire, for example, latitude, longitude, altitude, geocode, course, direction, heading, speed, UTC, date, and/or various other information/data. In one embodiment, the location module can acquire data, sometimes known as ephemeris data, by identifying the number of satellites in view and the relative positions of those satellites. The satellites may be a variety of different satellites, including LEO satellite systems, DOD satellite systems, the European Union Galileo positioning systems, the Chinese Compass navigation systems, Indian Regional Navigational satellite systems, and/or the like. Alternatively, the location information/data may be determined by triangulating the mobile computing entity's 300 position in connection with a variety of other systems, including cellular towers, Wi-Fi access points, and/or the like. Similarly, the mobile computing entity 300 may include indoor positioning aspects, such as a location module adapted to acquire, for example, latitude, longitude, altitude, geocode, course, direction, heading, speed, time, date, and/or various other information/data. Some of the indoor aspects may use various position or location technologies including RFID tags, indoor beacons or transmitters, Wi-Fi access points, cellular towers, nearby computing entities (e.g., smartphones, laptops) and/or the like. For instance, such technologies may include iBeacons, Gimbal proximity beacons, BLE transmitters, NFC transmitters, and/or the like. These indoor positioning aspects can be used in a variety of settings to determine the location of someone or something to within inches or centimeters.

The mobile computing entity 300 may also comprise a user interface device comprising one or more user input/output interfaces (e.g., a display 316 and/or speaker/speaker driver coupled to a processing element 308 and a touch screen, keyboard, mouse, and/or microphone coupled to a processing element 308). For example, the user interface may be configured to provide an application, browser, interactive user interface, dashboard, webpage, and/or similar words used herein interchangeably executing on and/or accessible via the mobile computing entity 300 to cause display or audible presentation of information/data and for user interaction therewith via one or more user input interfaces.

In one embodiment, the functionality described herein (and user interface) may be provided as a software application (e.g., an app) executing on the mobile computing entity 300. In such an implementation, the software application may be integrated with a variety of other software applications executing on the mobile computing entity 300 to gather information from the other applications. Such applications are typically designed to execute on mobile devices, such as tablets, smartphones, and/or virtual assistant devices. For example, the software application may be provided that executes on mobile device operating systems such as iOS®, Android®, or Windows®. These platforms typically provide frameworks that allow applications to communicate with one another and with particular hardware and software components of mobile devices.

Moreover, the user interface can comprise or be in communication with any of a number of devices allowing the mobile computing entity 300 to receive data, such as a keypad 318 (hard or soft), a touch display, voice/speech or motion interfaces, scanners, readers, or other input device. In embodiments including a keypad 318, the keypad 318 can include (or cause display of) the conventional numeric (0-9) and related keys (#, *), and other keys used for operating the mobile computing entity 300 and may include a full set of alphabetic keys or set of keys that may be activated to provide a full set of alphanumeric keys. In addition to providing input, the user input interface can be used, for example, to activate or deactivate certain functions, such as screen savers and/or sleep modes. Through such inputs, the mobile computing entity 300 can capture, collect, store information/data, user interaction/input, and/or the like.

The mobile computing entity 300 can also include volatile storage or memory 322 and/or non-volatile storage or memory 324, which can be embedded and/or may be removable. For example, the non-volatile storage or memory 324 may be ROM, PROM, EPROM, EEPROM, flash memory, MMCs, SD memory cards, Memory Sticks, CBRAM, PRAM, FeRAM, RRAM, SONOS, racetrack memory, and/or the like. The volatile storage or memory 322 may be RAM, DRAM, SRAM, FPM DRAM, EDO DRAM, SDRAM, DDR SDRAM, DDR2 SDRAM, DDR3 SDRAM, RDRAM, RIMM, DIMM, SIMM, VRAM, cache memory, register memory, and/or the like. The volatile and non-volatile storage or memory 322, 324 can store databases, database instances, database management system entities, data, applications, programs, program modules, scripts, source code, object code, byte code, compiled code, interpreted code, machine code, executable instructions, and/or the like to implement the functions of the mobile computing entity 300.

### Exemplary System Operations

The logical operations described herein may be implemented (1) as a sequence of computer implemented acts or one or more program modules running on a computing system and/or (2) as interconnected machine logic circuits or circuit modules within the computing system. The implementation is a matter of choice dependent on the performance and other requirements of the computing system. Accordingly, the logical operations described herein are referred to variously as states, operations, structural devices, acts, or modules. These states, operations, structural devices, acts, and modules may be implemented in software, in firmware, in special purpose digital logic, and any combination thereof. Greater or fewer operations may be performed than shown in the figures and described herein. These operations may also be performed in a different order than those described herein.

### End User Setup Module

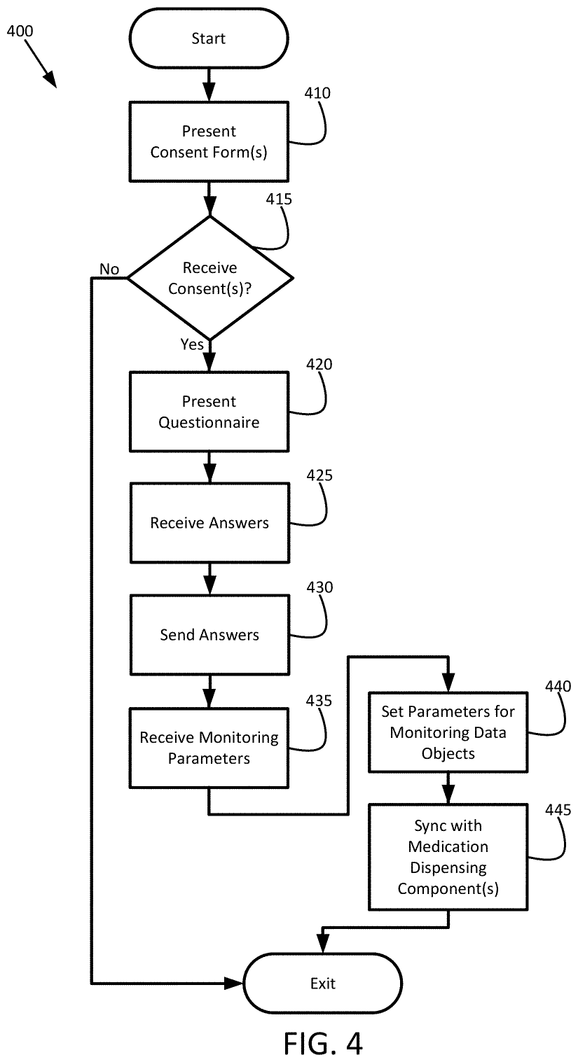

Turning now to FIG. 4, additional details are provided regarding a process flow for configuring an end user device 140 to perform end user monitoring according to various embodiments. FIG. 4 is a flow diagram showing an end user setup module for performing such functionality according to various embodiments of the disclosure. Here, the end user setup module may be a module found within a software application (e.g., an “app”) residing on the end user device 140 that may be, for example, a mobile device used by the end user. Accordingly, the mobile device may be a device such as a smartphone, tablet, smartwatch, laptop, and/or the like.

As previously noted, embodiments of the disclosure involve monitoring medication access requests made by the end user to gain access to medication that may be stored in a medication storage unit associated with a medication dispensing device 155 (e.g., in a storage unit such as a dispenser, container, bottle, etc.). In addition, embodiments may also be configured to monitor data objects (e.g., monitoring data objects) associated with factors that may influence whether or not the end user may experience a side effect as a result of taking a medication at a particular time. Accordingly, such monitoring may be performed via receiving monitoring data objects from the end user device 140, as well as receiving monitoring data objects from other sources. In some instances, such sources may require the end user's consent to obtain such data objects. For example, consent is often needed to access an individual's medical records. Therefore, the process flow 400 begins in various embodiments with the end user setup module presenting one or more consent forms to the end user to gain access to various information needed in monitoring the end user in Operation 410. Depending on the embodiment, a single form may be provided or multiple forms may be provided with respect to different sources of information. For example, a first consent form may be provided with respect to gaining access to the end user's medical records, while a second consent may be provided with respect to gaining access to credit card purchases from the end user's credit card provider. Those of ordinary skill in the art can envision multiple types of consents that may be asked of the end user to gain access to various information in light of this disclosure.

Accordingly, each consent form is presented to the end user on his or her device 140 along with a mechanism to provide consent (e.g., a button), and the end user indicates whether he or she consents to having the corresponding information monitored. Thus, in some embodiments, the end user can pick and choose what types of information can be accessed, as well as what types of objects (e.g., food purchases) can be monitored based at least in part on whether or not the end user provides consent to access the information and/or monitor the objects. In some instances, certain consents may be required by the end user to have his or her medication access requests monitored. Therefore, the end user setup module determines whether it has received the sufficient consent(s) from the end user in Operation 415.

If the appropriate consent(s) has been provided, then the end user setup module provides the end user with a questionnaire in Operation 420. Accordingly, the questionnaire may ask the end user questions on his or her current health condition, as well as past health condition. For example, the questionnaire may ask the end user what current medications he or she is taking. In addition, the questionnaire may ask the end user other health related information such as his or her current height and weight. The questionnaire may ask the end user if he or her currently has high blood pressure and/or has experienced high blood pressure in the past. In addition, the questionnaire may ask the end user about his or her family's health history. Further, the questionnaire may inquire about certain behaviors the end user may regularly participate in that may contribute to factors affecting the end user's taking of certain medications. For example, the questionnaire may ask the end user if he or she regularly consumes alcohol and if so, how much and how often. In addition, the questionnaire may ask the end user if he or she regularly exercises and if so, what types of exercise (e.g., cycling) the end user participates in. Here, the questionnaire may help to establish the an end user health profile for the user identifying the end user's current health, historical health, predisposition to certain health conditions, behavioral habits, and/or the like. As discussed further herein, information found within the end user health profile may be used in constructing other profiles for the end user. These other profiles may be considered subsets of the end user health profile. Once monitoring of the end user has begun, further input may be provided by the end user. For instance, the end user may be asked to routinely update his or her medical information (e.g., asked to routinely update his or her habits with respect to consuming certain foods).

The end user's medical history may also provide information used in particular embodiments in developing the end user health profile. Here, medical records may be obtained directly from the end user's caregivers (e.g., physicians) and/or the end user's electronic medical records (EMR). In addition, the end user's medical history may include clinical input provided by medical personnel who review the end user's records, as well as a whole genome sequence and/or standard interpretation of pre-defined risk alleles.

Finally, physiological data may also provide information used in particular embodiments in developing the end user health profile. As mentioned, one or more devices may be used to monitor various physiological conditions of the end user. For example, the end user may wear a fitness tracker that measures physiological conditions such as the end user's heart rate and/or physical activity (e.g., steps taken over a time period). Accordingly, such information may be provided as further input information used in developing the end user health profile. As detailed further herein, in some embodiments, measured physiological conditions may also be used in developing an end user factor profile for the end user. Therefore, in some instances, the questionnaire may be configured to automatically collect health information related to the end user. For example, the questionnaire can be configured to read the end user's current heart rate by using some monitoring device in communication with the end user setup module.

Accordingly, the end user setup module receives the answers to the questionnaire in Operation 425 and sends the answers to the predictive data analysis system 110 in Operation 430. The predictive data analysis system 110 receives the answers and uses the answers as one source of information in constructing the end user health profile. In particular embodiments, the predictive data analysis system 110 may use information found in the end user health profile once it has been constructed to identify various conditions and/or factors that may influence whether the user may experience side effects from taking a certain medication.

For example, the predictive data analysis system 110 may determine based at least in part on information found in the end user's health profile that the end user is currently diagnosed with borderline high blood pressure. Therefore, the predictive data analysis system 110 may identify a factor that the end user's current blood pressure should be monitored and considered when taking a particular medication prescribed to the end user since such medication is known to have a side effect of elevating an individual's blood pressure in some instances.

Accordingly, in particular embodiments, the end user setup module may receive one or more monitoring parameters associated with factors that have been identified for the end user in Operation 435. These monitoring parameters may indicate various monitoring data objects that should be gathered for the end user during the monitoring process, where the recommended monitoring data objects can be used in evaluating whether the end user should or should not take a dosage of a medication at a time when the end user is requesting access to the medication. For example, a relevant factor for the end user may be whether the end user has recently engaged in a physical activity (e.g., exercising). Therefore, a monitoring data object that may be gathered for the end user is motion/movement detected using some type of device such as a fitness tracker 150 that is in communication with the end user device 140. Thus, in Operation 440, the end user setup module may set the monitoring parameters needed to automatically collect motion/movement data detected by the fitness tracker 150.

In addition, the end user setup module may synchronize with any medication dispensing devices 155 that are configured to control access to stored medications and/generate one or more notifications to the end user upon detecting an end user attempt to access the stored medications, as well as any other devices that may be used in monitoring the end user such as virtual assistant devices 160, in Operation 445. Accordingly, a medication dispensing device 155 may itself be the storage unit and/or may be a device associated with the storage unit such as a lid or cap for the storage unit, lock for the storage unit, tag attached to the storage unit, and/or the like. Here, the medication dispensing device 155 and end user device 140 may be, for example, Bluetooth enabled in a manner that facilitates a Bluetooth connection between the two devices 140, 155. Once the mobile setup module has completed setting the monitoring parameters needed to gather the monitoring data objects for the relevant factors and synchronizing with the medication dispensing device(s) 155, the process for monitoring the end user's requests to access medications using the end user device 140 can begin.

### Monitoring Setup Module

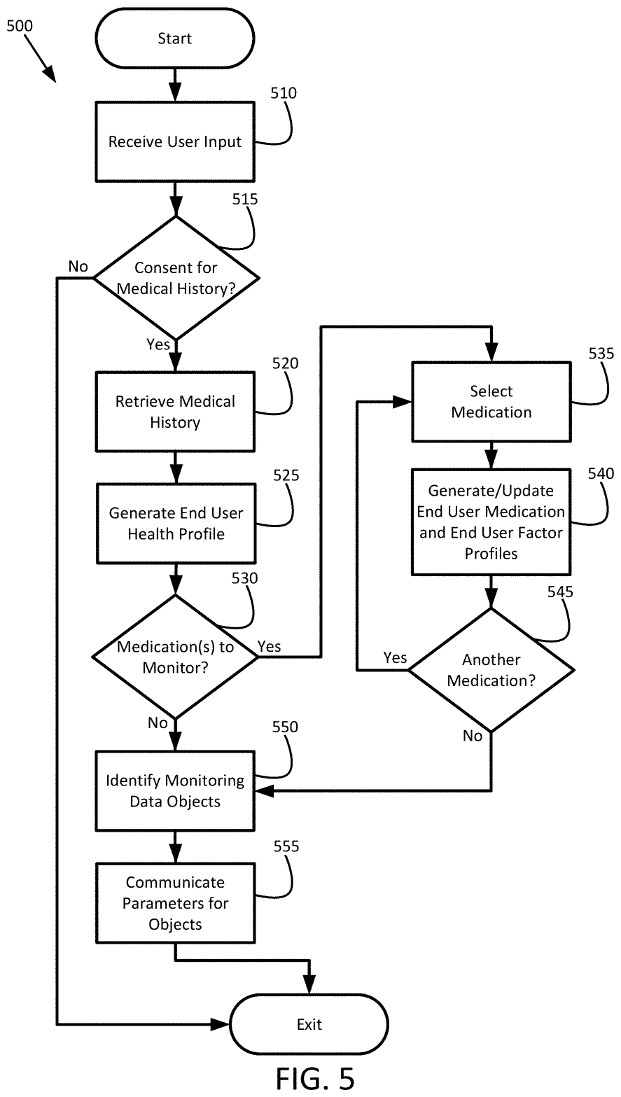

Turning now to FIG. 5, additional details are provided regarding a process flow for setting up the monitoring of an end user by the predictive data analysis system 110 according to various embodiments. FIG. 5 is a flow diagram showing a monitoring setup module for performing such functionality according to various embodiments of the disclosure. Specifically, the monitoring setup module may be used in setting up remote monitoring of the end user's medication access requests to gain access to medications by the predictive data analysis system 110.

The process flow 500 begins in various embodiments with the monitoring setup module receiving user input in Operation 510. As previously discussed, an end user who may wish to begin having his or her access requests to medications monitored may engage in a setup process that involves gathering information from the end user through the use of an instrument such as a questionnaire. The questionnaire may gather information on the end user with respect to the end user's current health state, as well as behavioral habits that may affect the end user's taking of certain medications.

In addition, the user input may include consent(s) provided by the end user to enable monitoring of certain information related to the end user (e.g., health records and/or food purchases), as well as consent(s) to gather information on the end user from various sources such as caregivers, pharmacist, credit card providers, wireless carrier providers, and/or the like. Therefore, the monitoring setup module determines whether consent to access the end user's medical records has been provided by the user in Operation 515.

If so, then the monitoring setup module retrieves the end user's medical records in Operation 520. As previously discussed, the end user's medical records may be obtained from various caregivers (e.g., physicians, nurses, in home personnel, and/or the like), pharmacists, and/or other sources such as health insurance providers for the end user, as well as the end user's EMR. Accordingly, the monitoring setup module may be configured to electronically retrieve the end user's medical records from various systems associated with the sources. Here, the monitoring setup module may use the end user's consent in gaining access to such information. In other instances, the monitoring setup module may be configured to initiate a process to gain access to the end user's medical records if such access cannot be gained immediately. For example, the monitoring setup module may initiate a request for a particular caregiver to gain access to the end user's health records held by the caregiver. Those of ordinary skill in the art can envision multiple mechanisms that can be employed in various embodiments to retrieve the end user's medical records in light of this disclosure.

At this point, the monitoring setup module generates the end user health profile for the end user in Operation 525. As already discussed, the end user health profile provides the end user's current health state and behavioral habits. Therefore, in various embodiments, the monitoring setup module uses the user input and medical history (e.g., medical records) in generating the end user health profile for the end user. It is noted that the end user health profile data may be updated from time to time in some embodiments based at least in part on updated information provided by the end user and/or updated medical information on the end user. For example, if the medical records for a caregiver may not be available during the initial setup process for the end user, then the end user health profile data may be updated once the medical records have been received. Further, the end user health profile may be updated based at least in part on a visit the end user makes to a caregiver, a new medication being prescribed to the end user, a change in an existing prescription, physiological data gathered on the end user during monitoring, and/or the like.

At this point, the monitoring setup module may determine whether the end user is currently taking (e.g., has currently been prescribed) any medications that are to be monitored in Operation 530. As discussed further herein, monitoring of the end user's use of such medications in various embodiments entails monitoring the end user's requests to gain access to the medications and, as a result, providing a confirmation to the end user as to whether he or she should take the medication at the time of the request. In addition, in some embodiments, monitoring of the end user's use of such medications may involving monitoring medical conditions that the end user may experience while taking the medications. This monitoring may entail determining whether such medical conditions may be due to medical problems the end user is experiencing or due to side effects the end user is experiencing as a result of interactions from taking multiple medications.

Accordingly, in various embodiments, a confirmation as to whether the end user should take a dosage of a medication may be based at least in part on conditions set for the end user's use of the medication. For example, a confirmation may be based at least in part on a condition that relates to the recommended frequency of medication intake by the end user (e.g., twice a day). As another example, a confirmation may be based at least in part on a condition that relates to taking the medication along with a meal. Such conditions may be defined, for example, by the prescribing physician and/or the pharmacist who fills the prescription for the end user. In some embodiments, these conditions may be identified based at least in part on instructions and/or restrictions set by the prescribing physician, as well as contraindications defined by a pharmacist and/or manufacturer of the medication.

In addition, the confirmation as to whether the end user should take a dosage of a medication may be based at least in part on factors that may influence whether the end user may experience a side effect as a result of taking the dosage of the medication. As used herein, “factors” may describe circumstances that exist at the time when the end user is attempting to take the dosage of the medication. For example, a condition may be set for a certain medication that the medication should not be taken with alcohol. Therefore, a factor that may be considered at the time when the end user is requesting access to the medication is whether the end user has recently consumed alcohol. Thus, factors may be tied to one or more monitoring data objects being tracked for the end user. For instance, in this example, a monitoring data object that may be tracked is the end user's purchases to determine whether the end user has recently purchased alcohol or the end user's location to determine whether the end user is visiting or has recently visited an establishment that sells alcohol.

In particular embodiments, these conditions and factors may be stored in an end user medication profile and/or an end user factor profile. Therefore, in these embodiments, if the monitoring setup module determines the end user is taking one or more medications that are to be monitored, then the monitoring setup module selects one of the medications in Operation 535. The monitoring setup module then generates the end user medication profile and the end user factor profile based at least in part on conditions and/or factors identified for the selected medications in Operation 540. In particular embodiments, the monitoring setup module performs this particular operation by invoking a profile module and in turn, the profile module identifies and stores the conditions and factors in the end user medication profile and the end user factor profile, respectfully. The monitoring setup module then determines whether the end user is taking another medication that is to be monitored in Operation 545. If so, then the monitoring setup module selects the next medication and generates the end user medication profile and the end user factor profile for the newly selected medication. It is noted that depending on the embodiment, a separate end user medication profile and/or end user factor profile may be generated and maintained for each medication being taken by the end user or a single end user medication profile and/or end user factor profile may be generated and maintained for all the medications taken by the end user. Therefore, the conditions and/or factors identified for a particular medication may be stored in separate profiles for a particular medication or appended to profiles maintained for all of the medications.

Once the monitoring setup module has processed all the medications for the end user, then the monitoring setup module identifies a set of monitoring data objects used in evaluating the identified factors for the medications in Operation 550. The set of monitoring data objects represents the different monitoring data objects that are to be gathered during the monitoring of the end user. The gathered monitoring data objects can then be used to identify factors that have occurred. Accordingly, occurrence of factors may influence the end user experiencing one or more side effects as a result of taking a particular medication. The monitoring setup module then communicates monitoring parameters for gathering one or more of the set of monitoring data objects to the end user device 140 in Operation 555. As previously discussed, the monitoring parameters are set in various embodiments to enable the end user device 140 in gathering various monitoring data objects. For example, a monitoring data object may be the end user's heart rate. Therefore, in this example, a parameter may be communicated to the end user device 140 that is to be set so that the end user's heart rate is recorded from a heart rate monitor 145 in communication with the end user device 140. Further, the monitoring setup module may setup the necessary parameters for one or more of the monitoring data objects that may involve other systems and devices other than the device 140 belonging to the end user. As a result, the end user device 140 may receive the monitoring parameters and take the necessary steps required to have the monitoring data objects collected accordingly. At this point, monitoring of the end user's access requests of his or her medications can begin.

It is noted that the monitoring setup process described in FIG. 5 can be re-run in various embodiments from time to time after monitoring of the end user begins. This may be done to update the medications, the end user health profile, the end user medication profile(s), the end user factor profile(s), and/or the set of monitoring data objects for the end user. Therefore, as the end user's current health state, medications, and behavioral habits may change over time, these parameters can be updated to reflect such.

### Profile Module

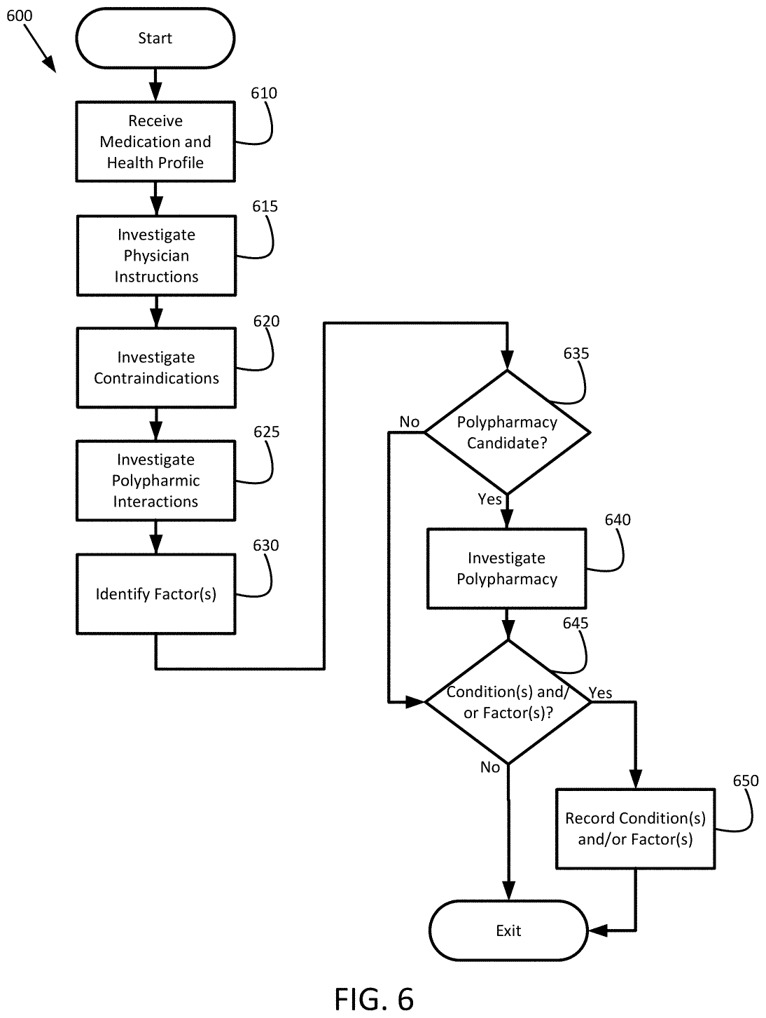

Turning now to FIG. 6, additional details are provided for setting the conditions and/or factors in profiles for a medication being taken by an end user according to various embodiments. FIG. 6 is a flow diagram showing a profile module for performing such functionality according to various embodiments of the disclosure. As previously mentioned, the profile module may be invoked by another module in particular embodiments to set the conditions and/or factors such as, for example, the monitoring setup module previously described. In some embodiments, the profile module may not necessarily be invoked by another module and may execute as a stand-alone module in other embodiments. For example, the profile module may be invoked in some embodiments as a result of a new medication being prescribed for an end user or an existing prescription for a medication being updated for the end user. For instance, the profile module may be invoked as a result of the end user being prescribed a new medication so that the appropriate profiles can be created and/or updated to reflect the conditions and/or factors for the new medication.

The process flow 600 begins with the profile module receiving the medication and the end user health profile for the end user in Operation 610. As previously mentioned, the end user health profile provides the end user's current health state and behavioral habits. Therefore, the end user medical profile may identify the medications the end user is currently taking, current and/or past medical conditions the end user is or has experienced, behavioral habits such as certain foods the end user likes to eat, and/or the like.

The profile module then investigates any instructions and/or restrictions that may have been set by the physician who has prescribed the medication to the end user in Operation 615. Here, in particular embodiments, such instructions and/or restrictions may be found in the end user health profile. However, in some embodiments, the profile module may be configured to query other sources such as the end user's EMR and/or query data (information) directly from the physician (e.g., system for the physician). Accordingly, the profile module may query these other sources to ensure the data being used to identify the physician's instructions and/or restrictions is current.

In addition, the profile module investigates any contraindications that may be associated with the medication in Operation 620. In general, a contraindication is a circumstance that serves as a reason to withhold a certain medical treatment because it may be harmful for an individual. For example, having a bleeding disorder is a contraindication for taking aspirin because treatment with aspirin may cause excess bleeding. Again, in particular embodiments, the profile module may be configured to query the end user health profile in identifying any applicable contraindications for the medication. For instance, the end user health profile may indicate the end user has a bleeding disorder. In addition, the profile module may query other sources to identify applicable contraindications such as the pharmacist who is filling prescriptions for the medication (e.g., the pharmacist's system) and/or the manufacturer of the medication (e.g., the manufacturer's system). Accordingly, the instructions, restrictions, and contraindications may serve as conditions placed on the end user's use of the medication.

Further, the profile module investigates any polypharmic interactions that may be associated with the medication in Operation 625. A polypharmic interaction is an interaction that may occur as a result of taking two or more medications. For example, combining fluoxetine and phenelzine can result in a central serotonin syndrome. While combining potassium chlorine and spironolactone can result in hyperkalemia. Here, the medication module may be configured to query one or more external sources such as, for example, PharmGKB's website and/or DrugBank's website to identify any known interactions that may exist for the medication. Accordingly, the profile module is configured in particular embodiments to generate or update a polypharmacy profile for the interactions identified for the medication. As detailed further herein, the polypharmacy profile may be used whenever an end user is requesting access to the medication to assist in identifying any interactions that may occur as result of taking the medication.

It is noted that in particular embodiments, the investigation carried out to identify known interactions for a medication may not necessarily be performed by the profile module, but instead may be performed by another module that is configured specifically to carry out this operation. This other module may be tasked to periodically generate and/or update polypharmacy profiles for various medications. However, having the profile module generate and/or update the polypharmacy profile for a medication can help to ensure such a profile exists whenever an end user is taking the medication and/or ensure the profile reflects the most up-to-date interactions. However, with that said, a polypharmacy profile may be maintained for each individual end user in some embodiments that is specific to that end user.

At this point, the profile module identifies any factors that may be relevant with respect to the medication and/or conditions in Operation 630. For example, the medication may be used for treating some type of cancer. Here, the end user's medical profile may indicate the end user has a habit of consuming grapefruit juice in the morning with breakfast. Grapefruit juice is known to cause an adverse interactions with some cancer medications. Therefore, a condition may have been defined requiring the end user to refrain from consuming grapefruit juice. However, although the end user may have been instructed not to consume grapefruit juice, the end user may forget the instruction and accidently purchase some grapefruit juice at the grocery store. Thus, the end user's habit of consuming grapefruit juice may be of relevance in setting up a factor used in monitoring the end user's use of the medication. In this particular example, the factor may involve determining whether the end user has purchased any grapefruit juice in the past week. Accordingly, a monitoring data object may be identified involving monitoring the end user's grocery purchases.

In particular embodiments, the profile module may employ a medication factors machine learning model in identifying the applicable factors for the end user. Here, information found in the end user health profile along with the applicable conditions may be provided as input to the model and the model may generate a medication factors data object for the end user where the medication factors data object provides a vector having a medication factor score for a number of factors that may be considered of interest to the medication. Therefore, in some embodiments, a different medication factors data object may be developed for different medications and/or conditions. Accordingly, each medication factor score represents a probability of the factor's relevance in determining whether to confirm the end user's use of the medication. Thus, the profile module may identify the relevant factors as those factors having a medication factor score exceeding a threshold.

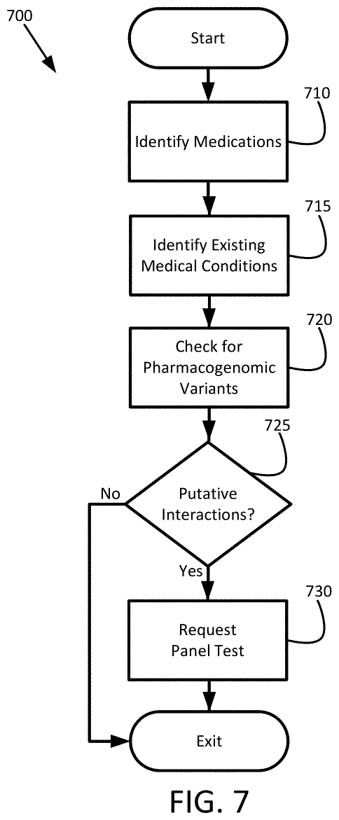

Once the profile module has identified the relevant factors, the profile module determines whether the end user is a polypharmacy candidate in Operation 635. A polypharmacy candidate is an end user who is currently taking multiple medications. If so, then the profile module conducts an polypharmacy investigation of the end user in Operation 640. As detailed further herein in FIG. 7, the polypharmacy investigation entails analyzing the end user's pre-existing medical conditions and side effects (e.g., medical conditions) that can result from an interaction between the medication and another medication the end user may be taking.

Accordingly, in various embodiments, a baseline may be established for the end user with respect to what medical conditions the end user may be experiencing prior to the end user beginning a medication and ongoing monitoring of the end user may then be conducted to identify any new medical conditions that may develop after the end user begins taking the medication. These new medical conditions may then be investigated in light of known interactions involving the medication and other medications the end user is taking to determine whether the new medical conditions are due to medical problems the end user is experiencing outside of taking the medication or as side effects due to an interaction. Such analysis and monitoring may be helpful in understanding medication interactions the end user may be experiencing, in addition to identifying medication redundancy and over-prescribing for the end user and preventing the end user from experiencing prescription cascade. Therefore, the polypharmacy investigation may help in establishing the baseline for the end user in evaluating any new medical conditions the end user may experience once he or she begins taking the medication.

At this point, the profile module determines whether any conditions and/or factors have been identified for the medication and end user in Operation 645. If so, then the profile module records the conditions and/or factors in Operation 650. As previously noted, this particular operation may involve the profile module generating an end user medication profile and an end user factor profile for the end user and particular medication or appending the identified conditions and/or factors identified for the medication to an existing end user medication profile and end user factor profile for the end user.

### Polypharmacy Module

Turning now to FIG. 7, additional details are provided for evaluating an end user who is a polypharmacy candidate according to various embodiments. FIG. 7 is a flow diagram showing a polypharmacy module for performing such functionality according to various embodiments of the disclosure. As previously mentioned, the polypharmacy module may be invoked by another module in particular embodiments to conduct the evaluation such as, for example, the profile module previously described. However, with that said, the polypharmacy module may not necessarily be invoked by another module and may execute as a stand-alone module in other embodiments. For example, the polypharmacy module may be invoked in some embodiments from time to time to updated the evaluation for an end user.

The process flow 700 begins with the polypharmacy module identifying the medications the end user is currently taking in Operation 710. Here, the polypharmacy module may perform this particular operation in various embodiments by querying one or more of the end user's profiles such as the end user health profile and/or the end user medication profile. In addition, the polypharmacy module identifies any existing medical conditions for the end user in Operation 715. Again, the polypharmacy module may perform this particular operation by querying one or more of the end user's profiles.

At this point, the polypharmacy module may query one or more external sources that provide various information on medications such as, for example, PharmGKB's website and/or DrugBank's website to identify any pharmacogenomic variants that may affect the end user's medications he or she is taking in Operation 720. A pharmacogenomic variant refers to a genetic variant an individual may have that may affect the individual's response to a medication. Here, the polypharmacy module may use a unique code (e.g., accession number) for the medication to query the generic drug name for the medication. Accordingly, the external sources may provide any known pharmacogenomic variants for the medication.

The polypharmacy module then determines whether any putative interactions may be suspected to exist between any of the medications the end user is taking in Operation 725. Here, the polypharmacy module may perform this operation by determining whether any two of the medications being taken by the end user have a similar pharmacogenomic variant. If so, then the polypharmacy module causes a panel test to be ordered for the end user to determine whether the end user has a genetic variation related to the similar pharmacogenomic variant in Operation 730.

Accordingly, the panel test can identify possible side effects the end user may experience as a result of taking the medication along with the other medications the end user is taking. As previously noted, a baseline may be established for the end user that describes what medical conditions the end user may be experiencing prior to the end user beginning a medication, and ongoing monitoring of the end user may then be conducted to identify any new medical conditions that may develop after the end user begins taking the medication. These new medical conditions may then be investigated in light of known interactions involving the medication and other medications the end user may be taking to determine whether the new medical conditions are due to medical problems the end user is experiencing outside of taking the medication or as side effects due to an interaction. Such analysis and monitoring may be helpful in understanding medication interactions the end user may be experiencing, in addition to identifying medication redundancy and over-prescribing for the end user and preventing the end user from experiencing prescription cascade.

Prescription cascade is a process whereby the side effects of a medication are misdiagnosed as symptoms of another medical problem, resulting in further medication being prescribed and further side effects and unanticipated medication interactions, which itself may lead recursively to further misdiagnoses and further symptoms. Therefore, by establishing a baseline for the end user, as well as recognizing what side effects the end user may experience due to interactions between medications the end user is taking, the problem of prescription cascade may be avoided for the end user. If any new medications are added to the end user's daily intake, monitoring can then be performed of the end user for any new conditions from a list of the most common medication interactions and adverse drug events (e.g., available via the U.S. Food and Drug Administration).

In addition, with the end user's consent, a summary care record may be created and de-identified that may be used to populate a repository of polypharmacy data. Accordingly, this data can be made available to medical and pharmacology researchers to provide key, quality data to better understand polypharmacy. In addition, the summary care record could be provided to the end user caregiver(s) to provide opportunities for data-driven deprescribing.

### End User Monitoring Module

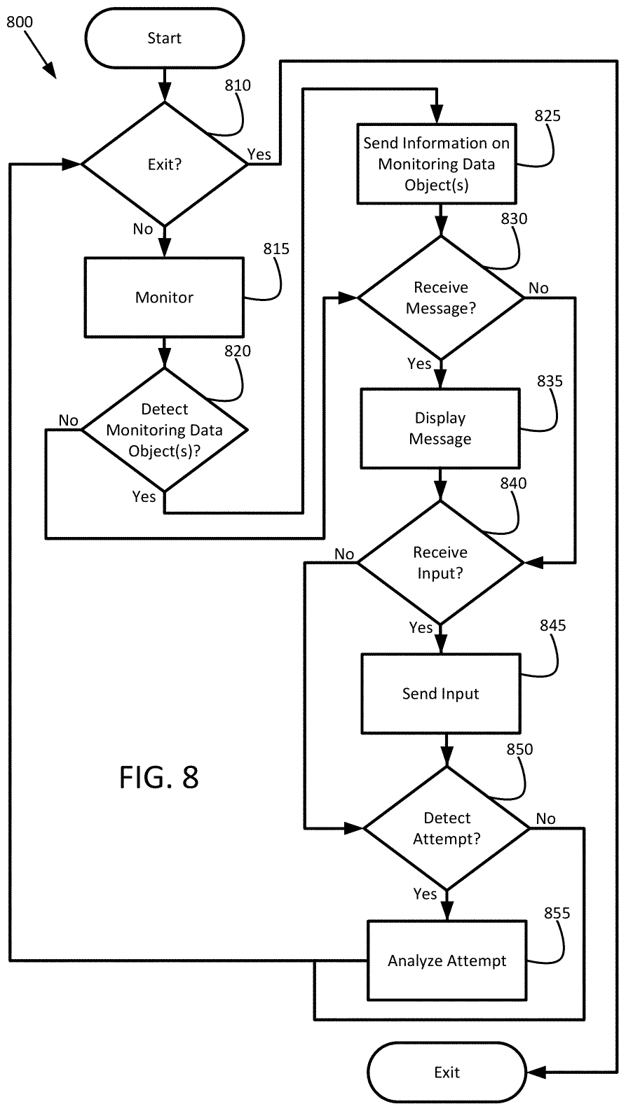

Turning now to FIG. 8, additional details are provided regarding a process flow for gathering monitoring data objects and monitoring for access requests to medications for an end user who is using a device 140 such as, for example, a mobile device according to various embodiments. FIG. 8 is a flow diagram showing an end user monitoring module for performing such functionality according to various embodiments of the disclosure. Similar to the end user setup module, the end user monitoring module may be a module found within a software application (e.g., an “app”) residing on the device 140 used by the end user. As discussed below, the end user monitoring module is configured to receive monitoring data objects from various sources (e.g., a fitness tracker 150 being worn by the end user), as well as attempt data objects as a result of the end user generating (e.g., triggering) a medication access request for a particular medication. Accordingly, the end user monitoring module is configured in various embodiments to send the monitoring data objects to the predictive data analysis system 110 and initiate an analysis for the each of the attempt data objects received.

The process flow 800 begins with the end user monitoring module determining whether to exit in Operation 810. For instance, the end user may have decided to end the monitoring of his or her access and use of medications and as a result, discontinues use of the end user monitoring module and/or software application by shutting one and/or the other off. If the end user monitoring module determines not to exit, then the module begins monitoring in Operation 815.

Accordingly, the mobile monitoring module may receive monitoring data objects from various sources such as the end user device 140, itself, and/or from various monitoring devices in communication with the device 140. In addition, the end user may directly enter a monitoring data object into the software application. For example, the software application may enable the end user to scan in receipts from purchases and/or identify food products he or she has consumed. Therefore, the end user monitoring module may receive monitoring data objects that may represent several different types of data, such as, for example, physiological data gathered on the end user, purchasing data, location data, event data from the end user's calendar, and the like.

Therefore, the end user monitoring module determines whether one or more monitoring data objects have been detected (e.g., communicated) in Operation 820. If so, then the module sends the monitoring data object(s) to the predictive data analysis system 110 in Operation 825. In turn, the predictive data analysis system 110 may identify one or more factors associated with the one or more monitoring data objects and send a message to confirm occurrence of the factor(s) with the end user. If this is the case, then the end user monitoring module may determine whether it has received such a message in Operation 830, and if so, communicate (e.g., display) the message to the end user in Operation 835. Accordingly, the end user may respond to the message (e.g., confirm occurrence of the factor(s)) and the end user monitoring module determines whether it has received the end user's response (e.g., confirmation) in Operation 840. If so, then the end user monitoring module sends the response to the predictive data analysis system 110 in Operation 845.

The predictive data analysis system 110 and end user may exchange information (e.g., messages) that is not necessarily related to a particular factor that has been detected. For example, the software application may enable the end user to request information on the various conditions set for a particular medication the end user is taking. Such information may help the end user to adhere these conditions. Therefore, in this example, the end user monitoring module may determine that the end user has inputted a request for such information and as a result, send the request to the predictive data analysis system 110.

The end user monitoring module determines whether an attempt data object has been received in Operation 850. As previously noted, the end user device 140 may receive an attempt data object as a result of the end user generating a medication access request for a particular medication. Here, the medication may be stored in some type of storage unit (e.g., a medication dispenser) and the storage unit may be associated with a medication dispensing device 155. Accordingly, in some embodiments, the medication dispensing device 155 and/or the end user device 140 may be configured to detect the medication access request.

For example, the medication dispensing device 155 may be configured to detect movement signaling that the end user is handling the storage unit in an attempt to gain access to the medication in the storage unit. Such movement may be interpreted as the end user communicating a medication access request and, as a result, the medication dispensing device 155 may send (e.g., transmit) an attempt data object to the end user device 140. In another example, the medication dispensing device 155 and/or the end user device 140 may detect one another within a distance threshold from each other. For instance, the medication dispensing device 155 may transmit a signal (e.g., via an RFID tag) that the end user device 140 detects when the end user moves to within a threshold distance from the medication dispensing device 155. This detection can serve as a communication of a medication access request from the end user. Those of ordinary skill in the art can envision other mechanisms that may be used for recognizing the end user communicating a medication access request.

Accordingly, if the end user monitoring module determines an attempt data object has been received, then the end user monitoring module analyzes the attempt being made to access the medication in Operation 855. Here, the analysis is carried out in various embodiments with the assumption that the end user is requesting to access the medication for the purpose of taking a dosage of the medication. Therefore, the analysis is conducted as to whether the end user should or should not take the dosage of the medication at the time of the request.

As discussed further herein, the analysis may be based at least in part on the various applicable conditions and/or factors that may influence whether the end user may experience an adverse side effect if he or she were to take the dosage of the medication. In addition, the analysis may be based at least in part on whether an interaction may result from the end user taking the dosage of the medication. For example, an interaction may be known to exist between two different medications if they are taken within close proximity with respect to time to each other. Here, the end user may be currently taking both medications and, as a result, the analysis may take into consideration this known interaction in determining whether the end user should take the dosage of the medication.

In particular embodiments, to conduct the analysis on the attempt data object, the end user monitoring module engages (e.g., by sending a request to) the predictive data analysis system 110 and the system 110 conducts the analysis for the attempt data object and sends back a confirmation as to whether the end user should or should not take the dosage of the medication. As discussed further herein, in some instance, the analysis conducted by the predictive data analysis system 110 may involve generating an inferred prediction as to whether or not the end user should or should not take the dosage of medication.

Accordingly, in some embodiments, the end user monitoring module may be configured to invoke a local module on the end user device 140 that may carry out the analysis instead of engaging the predictive data analysis system 110. The end user monitoring module may be configured in such a manner to allow for the analysis to be carried out even when the end user device 140 is unable to communicate with the predictive data analysis system 110. For example, the end user may be in a location without internet service or the end user's internet service may be temporarily unavailable. Here, in particular embodiments, the functionality and information (e.g., the different profiles) found on the end user device 140 may be configured to perform an abbreviated version of the analysis to save memory storage on the device 140. For example, the end user factor profile may not be stored on the end user device 140 or a redacted version of the end user factor profile may be stored on the end user device 140 and used when a local analysis is performed to save memory space. Therefore, although a complete analysis may not be performed in some embodiments when the end user device 140 is unable to communicate with the predictive data analysis system 110, at least some level of analysis may still be performed to help ensure the end user is properly taking his or her medications.

### Attempt Confirmation Module

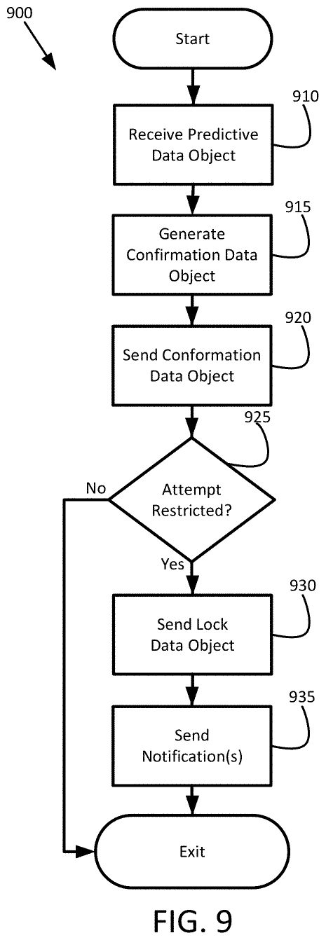

Turning now to FIG. 9, additional details are provided regarding a process flow for generating a confirmation data object in regard to a medication access request made by an end user who is attempting to access a medication according to various embodiments. FIG. 9 is a flow diagram showing an attempt confirmation module for performing such functionality according to various embodiments of the disclosure. For example, the attempt confirmation module may be a module found within a software application (e.g., an “app”) residing on the device 140 used by the end user.

Accordingly, the attempt confirmation module may be invoked in various embodiments as a result of receiving a predictive data object. Here, the predictive data object may indicate an inferred prediction about various applicable conditions, factors, and/or interactions associated with the end user taking a dosage of the medication he or she is requesting (e.g., attempting) to access. For example, in particular embodiments, the predictive data object may be a predictive polypharmacy data object that indicates an inferred prediction about polypharmacy compatibility of the polypharmacy profile associated with the medication the end user is requesting to access and the end user factor profile for the end user. As now described, the predictive data object is used to set a confirmation data object that can then be used to communicate to the end user a recommendation about whether he or she should take a dosage of the medication he or she is requesting to access.

Therefore, the process flow 900 begins with the attempt confirmation module receiving the predictive data object in Operation 910. In particular embodiments, the predictive data object is provided by the predictive data analysis system 110 as a result of the system 110 conducting an analysis after receiving a request from the end user device 140 based at least in part on an attempt data object for the end user's access request. However, as previously noted, the end user device 140 may be configured in some embodiments to perform the analysis locally. Therefore, the predictive data object may be provided by the end user device 140 in some instances.

The attempt confirmation module then generates a confirmation data object based at least in part on the predictive data object in Operation 915. In various embodiments, the confirmation data object indicates a recommended response to the attempt data object received based at least in part on the end user's access request for the medication. Therefore, the confirmation data object may indicate that the end user should or should not take a dosage of the medication he or she is requesting to access. In some embodiments, the confirmation data object may also indicate that a determination could not be reached from the analysis as to whether or not the end user should take the dosage of the medication (e.g., the confirmation data object may indicate an undetermined state). For example, the system and/or device conducting the analysis may not have been able to access data needed in conducting the analysis. Furthermore, the confirmation data object may indicate a reason for the determination.

Therefore, once the attempt confirmation module has generated the confirmation data object, the attempt confirmation module sends (e.g., transmits) the confirmation data object to one or devices in Operation 920. For example, the attempt confirmation module may send the confirmation data object to a medication dispensing device 155 associated with a storage unit used for storing the medication. In turn, the medication dispensing device 155 may generate one or more dynamic electronic user notifications based at least in part on the confirmation data object that are configured to communicate to the end user whether or not to take the dosage of the medication. For instance, the one or more dynamic electronic user notifications may comprise audiovisual data such as light that is illuminated in various colors to convey to the user whether or not to take the dosage of the medication. For example, the medication dispensing device 155 may illuminate the color green to confirm (approve) the end user's taking of the dosage of medication, the color red to restrict (deny) the end user's taking of the dosage of medication, and the color yellow to indicating a determination could not be reach as whether the end user should take the dosage of the medication.

Accordingly, the attempt confirmation module may send the confirmation data object to other devices to communication the recommended response to the end user such as a virtual assistant device 160. Here, the virtual assistant device 160 may provide electronic user notifications in the form of audio data, visual data, and/or a combination of both. In some embodiments, voice recordings of family members may be used as electronic user notifications. This may be especially beneficial for older end users and/or end users who may have dementia, as research has shown better response from using such an approach. The same approach can be used with respect to videos of family members.

In some embodiments, the attempt confirmation module may be configured to determine whether the confirmation data object (or predictive data object) indicates a restriction/rejection of the end user from taking the dosage of the medication in Operation 925. If so, then the attempt confirmation module may send a locking data object to the medication dispensing device 155 in Operation 930. Accordingly, the medication dispensing device 155 may be configured with a locking mechanism that allows the medication dispensing device 155 to lock the storage unit holding the medication to prevent the end user from accessing the medication. Therefore, responsive to receiving the locking data object, the medication dispensing device may lock the storage unit. In some embodiments, the conformation data object may also be used to perform this functionality instead of sending a locking data object.

Finally, in particular embodiments, the attempt confirmation module may send one or more notifications in response to the confirmation data object indicating a restriction/rejection of the end user from taking the dosage of the medication in Operation 935. For example, the attempt confirmation module may send notification(s) to a caregiver and/or family member of the end user to inform them that the end user made a non-conformant request to access medication. This may help those who receive such notifications to become aware of the end user's non-conformance and possibly address the non-conformance. Accordingly, the attempt confirmation module may be configured to send the notifications using various forms of communication such as emails, text messages, voice calls, and/or the like.

### Remote Monitoring Module

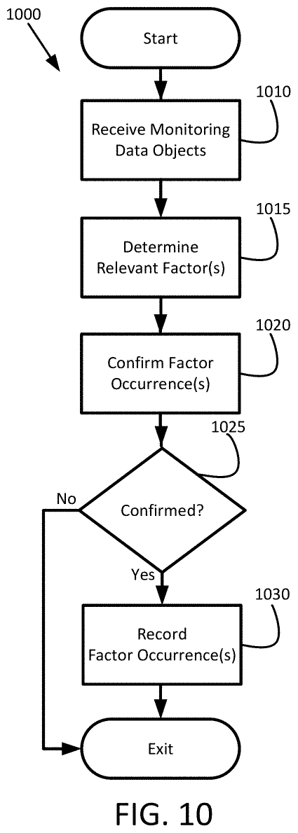

Turning now to FIG. 10, additional details are provided regarding a process flow for monitoring an end user remotely according to various embodiments. FIG. 10 is a flow diagram showing a remote monitoring module for performing such functionality according to various embodiments of the disclosure. For example, the remote monitoring module may be hosted within the predictive data analysis system 110 previously discussed. Here, the predictive data analysis system 110 may invoke the remote monitoring module based at least in part on one or more monitoring data objects received for the end user.

For instance, one or more trigger events may be defined that are used to identify occurrences of factors for the end user that should be monitored/identified based at least in part on one or more monitoring data objects that are received for the end user. For example, a monitoring data object (e.g., a GPS data object) may be received that indicates the end user is in the proximity of a restaurant. Here, the end user's location may serve as a trigger event to invoke the remote monitoring module. Accordingly, the predictive data analysis system 110 may gather one or more monitoring data objects that may be related such as, for example, purchasing information gathered for a time interval when the end user is/was detected to be located within the proximity of the restaurant, and invoke the remote monitoring module to analyze the one or more monitoring data objects. Those of ordinary skill in the art can envision other trigger events that may be defined and used in light of this disclosure.

Therefore, the process flow 1000 begins with the remote monitoring module receiving the one or more monitoring data objects in Operation 1010. In addition, information may be provided to the remote monitoring module identifying the particular end user. The remote monitoring module then determines the relevant factor(s) for the one or more monitoring data objects in Operation 1015. For example, in particular embodiments, the remote monitoring module may reference the end user factor profile for the end user to identify the relevant factor(s) for the one or more monitoring data objects. Here, the various monitoring data objects may be identified in the end user factor profile along with their corresponding factors. While in other embodiments, the remote monitoring module utilizes a lookup table that is maintained to identify what monitoring data objects are used for various factors. In some embodiments, the same table may be used in identifying the monitoring data objects to monitor for an end user during the setup process.

In particular embodiments, the remote monitoring module may be configured to confirm occurrence of the factor(s) in Operation 1020. For example, this operation may involve the remote monitoring module having a message sent to a device 140 of the end user and displayed via a software application hosted on the device 140. The end user may then confirm or deny that the factor(s) have occurred. For example, one of the factors identified to have occurred may describe whether the end user has participated in a physical activity (e.g., exercise) that may raise the end user's heart rate for a period of time. Here, the factor may have been identified based at least in part on one or more monitoring data object indicating the end user engaging in rapid movement (e.g., running). Therefore, the end user may confirm that he or she did in fact participate in a physical activity. If the remote monitoring module determines the end user has confirmed at least one of the factors identified to have occurred in Operation 1025, then the remote monitoring module records the factor occurrences in the end user factor profile for the end user in Operation 1030.

It should be noted that depending on the circumstances and the type of factor involved, the one or more monitoring data objects used in identifying the occurrence of a particular factor may be received in real time, after the fact, or before the factor has occurred in various embodiments. For instance, if the end user is participating in a physical activity (e.g., exercising) and this factor is detected by a fitness tracker 150 being worn by the end user, then a monitoring data object on the factor may be provided to the remote monitoring module in real time while the end user is participating in the activity. While in other instances, the one or more monitoring data objects for a particular factor may be communicated after the fact. For example, the end user may have visited a restaurant and purchased dinner using his or her credit card. Here, the visit may be detected based at least in part on purchasing information received from the end user's credit card provider after the purchase and therefore, the monitoring data object for the related factor (e.g., consumption of alcohol) may be communicated after the factor has occurred. Finally, in some instances, the one or more monitoring data objects for a particular factor may be communicated prior to the factor occurring. For example, the end user may be scheduled for an appointment he or she needs to drive to that is shown on the end user's electronic calendar that is being monitored. Here, the appointment on the calendar may be detected prior to the end user driving to the appointment and, therefore, the monitoring data object for the related factor (e.g., operating of motor vehicle) may be communicated prior to the factor occurring.

In addition, although not shown in FIG. 10, the remote monitoring module may conduct an analysis of the factor occurrences for an end user to determine whether the end user may be developing factor patterns that may contribute to undesirable side effects for the end user. For example, the end user may have developed a habit of taking a daily walk just before the end user is scheduled to take a particular medication that results in the end user regularly reaching a body temperature above a risk level for taking the medication. Here, the remote monitoring module may be configured to use one or more models (e.g., one or more machine learning models) for identifying such patterns. For example, in particular embodiments, the end user health profile, the end user medication profile, and/or the end user factor profile may be processed using one or more machine learning models to produce predictions (e.g., probabilities) on whether the end user may be developing patterns with respect to the factors associated with the medication.

For instance, the one or more machine learning models may generate a factors pattern data object for the end user where the factors pattern data object provides a vector having a factor pattern score for each of the factors associated with the medication. Accordingly, each factor pattern score may represent a probability of the end user having developed a pattern with respect to the factor that may contribute to undesirable side effects for the end user. Here, a factor pattern score exceeding a threshold may indicate the end user has developed a pattern for the factor. Accordingly, the recognized pattern(s) may then be communicated to the end user so that he or she may adjust the pattern(s) to avoid having a problem when he or she is scheduled to take the medication.

### Attempt Evaluation Module

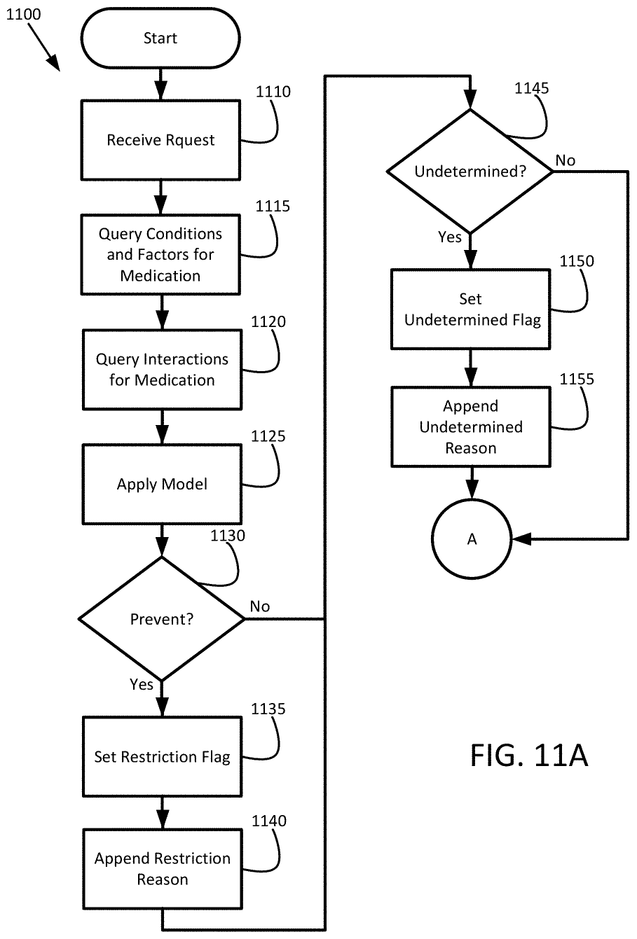

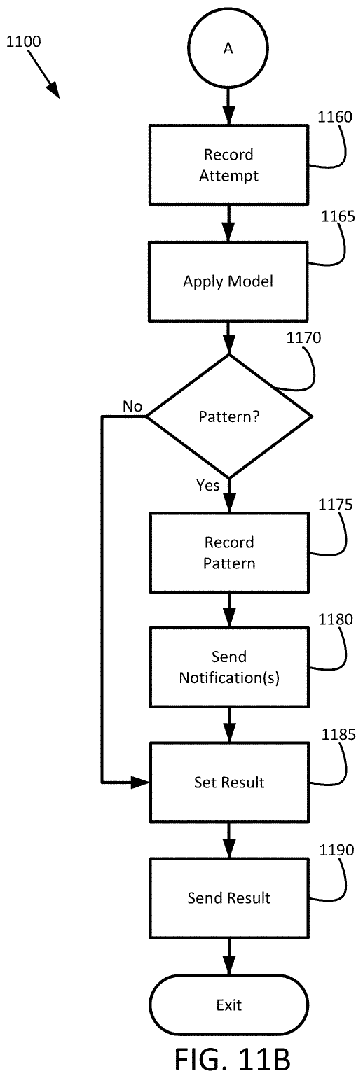

Turning now to FIGS. 11A and 11B, additional details are provided for evaluating an end user's attempt (request) to access a medication according to various embodiments. FIGS. 11A and 11B are a flow diagram showing an attempt evaluation module for performing such functionality according to various embodiments of the disclosure. For example, the attempt evaluation module may be hosted within the predictive data analysis system 110 previously discussed. Here, the predictive data analysis system 110 may invoke the attempt evaluation module based at least in part on a received a request due to an attempt data object for the end user.

Turning first to FIG. 11A, the process flow 1100 begins with the attempt evaluation module receiving the request that is generated based at least in part on an attempt data object in Operation 1110. Accordingly, in some embodiments, the attempt data object may be sent as the request. As previously discussed, the attempt data object may be generated in various embodiments as a result of the end user requesting (attempting) to access medication. For example, in particular embodiments, the end user may trigger a medication access request by physically handling and/or moving within a threshold distance to a medication dispensing device 155 associated with a storage unit used for storing the medication. As a result, the medication dispensing device 155 may generate the attempt data object and transmit the attempt data object to the end user device 140. While in other embodiments, the end user device 140 may detect the medication dispensing device 155 within a threshold distance and as a result generate the attempt data object. Accordingly, the end user device 140 may then send (e.g., transmit) the attempt data object as the request to the predictive data analysis system 110.

Accordingly, the request may identify the end user and the medication he or she is attempting to access. Therefore, upon receiving the request, the attempt evaluation module queries the conditions and/or factors for the medication in Operation 1115. The conditions and/or factors may also be specific to the end user in addition to the medication. Therefore, in particular embodiments, the attempt evaluation module may query the conditions and/or factors by querying the end user medication profile and end user factor profile for the end user. These profiles can be used for storing the conditions and factors that are applicable to the end user's use of the medication, as well as for storing data describing the occurrences of the factors. For example, a condition for the medication may be related to a recommended frequency as which the end user is to take the medication (e.g., twice a day) and a factor may be based at least in part on the monitoring of the end user's taking of the medication to determine the actual frequency of medication intake. Therefore, the profiles may indicate this condition and factor for the medication and end user, as well as occurrences detected of the end user taking the medication.

Next, the attempt evaluation module queries the known interactions for the medication in Operation 1120. Similar to the conditions and factors, the attempt evaluation module may carry out this operation in particular embodiments by querying the polypharmacy profile for the medication. As previously discussed, the polypharmacy profile may be common with respect to all end users or may be specific to the end user. In other words, a single polypharmacy profile may be maintained for the medication and used for every end user or individual polypharmacy profiles may be maintained for the medication for each end user. In some embodiments, a polypharmacy profile is maintained for a group of end users, such as a demographically-defined group of end users.

At this point, the attempt evaluation module processes the end user medication profile, the end user factor profile, and/or the polypharmacy profile using a prediction machine learning model (e.g., a polypharmacy prediction machine learning model) in Operation 1125. In particular embodiments, the prediction machine learning model may be a rule-based machine learning model configured to infer compatibility of the end user taking a dosage of the medication based at least in part on conditions, occurrences of factors, and interactions that may occur as a result of taking the medication. For example, the prediction machine learning model may be configured to infer that the end user can safely take a dosage of the medication based at least in part on compatibility of conditions placed on the end user, interactions that may occur, and occurrences (or lack thereof) of one or more factors that may have been detected via one or more monitoring data objects. In some embodiments, the prediction machine learning model may be a trained machine learning model that is configured to process the end user medication profile, end user factor profile, and/or polypharmacy profile to generate an inferred prediction about compatibility of the end user taking a dosage of the medication at the time the medication access request is made.

For example, in one particular embodiment, the prediction machine learning model may be a polypharmacy prediction machine learning model. Here, the machine learning model may be configured to generate an inferred prediction about polypharmacy compatibility of the polypharmacy profile for the medication and the end user factor profile for the end user. Thus, the model provides an inferred prediction on the compatibility between interactions that may occur for the medication and occurrences of factors found in the end user factor profile that may indicate an interaction can occur. For example, an interaction may be recorded for a first medication when taken with a second medication that the end user is currently prescribed. In this instance, the end user's physician may have issued a restriction requiring that the first medication not to be taken within three hours of the second medication. Accordingly, this restriction may serve as a condition placed on the end user (e.g., indicated in the end user medication profile) and an associated factor related to the tracking of the end user's administering of the two medications is generated. Therefore, the polypharmacy prediction machine learning model in this example may generate an inferred prediction based at least in part on the compatibility between the interaction found in the polypharmacy profile for the medication and occurrences of the end user taking the two medications as indicated in the end user factor profile.

Other configurations of the prediction machine learning model may be used in other embodiments to generate an inferred prediction about compatibility with respect to the end user taking a dosage of the medication at the time of the medication access request as those of ordinary skill in the art can envision in light of this disclosure. In addition, the machine learning model may comprise multiple models (e.g., an ensemble of models) in various embodiments to generate the inferred prediction about compatibility.

Once the prediction has been generated, the attempt evaluation module in particular embodiments determines whether the prediction about compatibility indicates the end user should be advised not to take and/or prevented from taking the dosage of medication in Operation 1130. In other words, the prediction may indicate non-compatibility with respect to the end user taking the dosage of medication at the time of the medication access request. If this is the case, the attempt evaluation module sets a restriction flag in Operation 1135. Accordingly, the restriction flag may be set in a data object that is returned to the end user device 140. In addition, in some embodiments, the attempt evaluation module appends a reason for the restriction in Operation 1140. For instance, in the example involving the interaction of the two medications, the reason may indicate that the second medication may have been taken within the three hour of the medication access request.

In addition, the attempt evaluation module in particular embodiments determines whether the predication about compatibility is undetermined (inconclusive) in Operation 1145. Here, the prediction machine learning model may be configured to provide a confidence measure (e.g., a value) along with prediction. This confidence measure indicates a level of confidence the model has in the prediction. Therefore, the attempt evaluation module may be configured to determine whether the confidence measure meets a threshold in some embodiments. If not, then the attempt evaluation module may determine the prediction about compatibility is undetermined. If this is the case, then then attempt evaluation module sets an undetermined flag in Operation 1150. In addition, the attempt evaluation module in some embodiments may append a reason for the undetermined state in Operation 1155.

Turning now to FIG. 11B, the process flow 1100 continues with the attempt evaluation module recording the attempt in Operation 1160. In addition, in particular embodiments, the attempt evaluation module performs an analysis to determine whether the end user may be experiencing non-adherence and/or adherence patterns with respect to the conditions placed on the end user in taking the medication. For example, the end user may be taking insulin and the end user may have developed a habit of not taking his or her insulin after drinking alcohol or not titrating the insulin does to carbohydrate intake. The attempt evaluation module may make use of one or more pattern recognition models, such as machine learning models and/or multivariate models, in identifying such patterns. These models may be configured in a similar fashion to the other models described herein.

For example, in some embodiments, the one or more pattern recognition models may be machine learning modules configured to process the end user's attempts to generate predictions (e.g., probabilities) on whether the end user is experiencing non-adherence and/or adherence patterns with respect to conditions placed on the end user in taking the medication. For instance, the one or more machine learning models may generate a conditions pattern data object for the end user where the conditions pattern data object provides a vector having a condition pattern score for each of the conditions placed on the end user in taking the medication. Accordingly, each condition pattern score may represent a probability of the end user having developed a pattern of non-adherence and/or adherence with respect to the condition. Specifically, a condition pattern score exceeding a threshold may indicate the end user has developed a pattern of not adhering or adhering to the condition. For example, a condition pattern score over a certain threshold may indicate the end user has developed a pattern of not adhering to the condition and a condition pattern score under a certain (different) threshold may indicate the end user has developed a pattern of adhering to the condition.

Accordingly, any patterns identified may be communicated to the end user and/or other individuals such as the end user's caregiver(s) and/or family members. Such notifications may help to identify and correct any non-conforming patterns the end user may have developed, as well as reinforce any conforming patterns the end user may have developed. For example, a notification may be sent to the end user (e.g., end user device 140) in recognition of a pattern of the end user taking his or her insulin at the correct designated times that states “You have been regularly adhering to your insulin schedule. Keep up the good work!” In some embodiments, reminder notifications may also be sent to promote conforming behavior.

Therefore, if the attempt evaluation module is configured to perform such an analysis, the attempt evaluation module processes the end user's history of attempts (e.g., reasons for conformance/non-conformance) in Operation 1165. The attempt evaluation module then determines whether any pattern was recognized in Operation 1170. If so, then the attempt evaluation module records the pattern and sends the appropriate notification(s) in Operations 1175 and 1180.

At that point, the attempt evaluation module sets the result for the analysis to determine whether the end user may be experiencing non-adherence and/or adherence patterns with respect to the conditions placed on the end user in taking the medication in Operation 1185. As already discussed, the analysis may indicate (e.g., generated an inferred prediction about compatibility) a conformance pattern related to the end user taking the dosage of medication, a non-conformance pattern related to the end user taking the dosage of medication, and/or that a conformance/non-conformance pattern could not be determined with a required degree of certainty. As a result, the attempt evaluation module may have set flags accordingly. Therefore, the attempt evaluation module may set the result for the analysis based at least in part on the set flags. Accordingly, in various embodiments, the result may then be provided as a data object that is returned to the end user device 140 and/or other devices such as a virtual assistant device 160, the medication dispensing device 155, etc. For example, the attempt evaluation module may set a predictive polypharmacy data object in embodiments in which the attempt evaluation module makes use of the polypharmacy prediction machine learning model. Once set, the attempt evaluation module sends the result in Operation 1190. As previously discussed, the result may then be communicated to the end user through one or more electronic user notifications via the medication dispensing device 155, the end user device 140, and/or another device such as a virtual assistant device 160.

As previously noted, in particular embodiments, the end user device 140 and/or another device such as the medication dispensing device 155 and/or a virtual assistant device 160 may be configured to conduct a process flow the same or similar to the process flow 1100 described in FIGS. 11A and 11B. The end user device 140 or other device may be configured in this fashion so that an analysis can be conducted for a medication access request generated by the end user without having to communicate with the predictive data analysis system 110. Such a capability may be advantageous in various embodiments because an analysis can still be conducted locally in instances when connectivity between the end user device 140 and the predictive data analysis system 110 may be lost. While in some embodiments, the end user device 140 and/or another device may be configured to solely conduct the analysis without the need for the predictive data analysis system 110.

### Examples of User Notification Messages

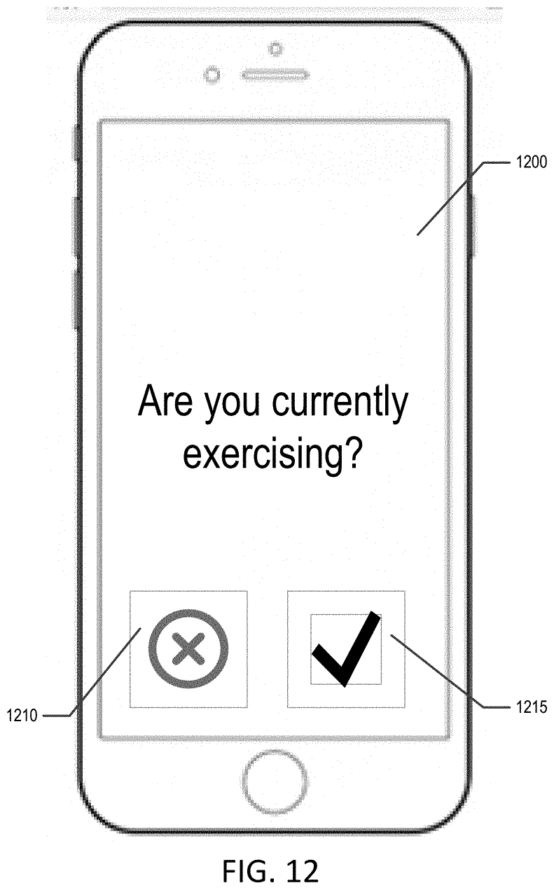

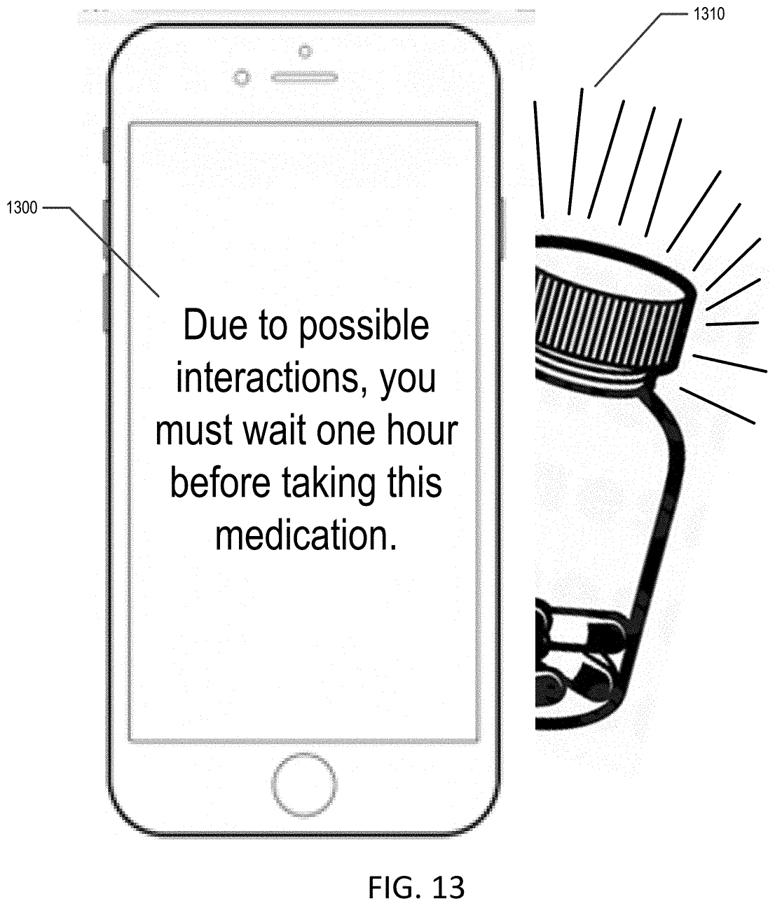

FIGS. 12 and 13 provide two examples of messages that may be displayed on an end user device 140 in accordance with various embodiments. Specifically, FIG. 12 provides an example of a message that may be displayed on an end user device 140 asking the end user whether he or she is participating in a particular activity that may serve as a factor. Accordingly, the screen 1200 of the device 140 is displaying a message asking the end user if he or she is currently exercising. Such a message may be displayed to confirm the occurrence of the factor after detecting one or more monitoring data objects indicating the factor. For example, the end user device 140 may be communicating with a fitness tracker 150 being worn by the end user that tracks physical activity. The message may be generated in response to an activity notification from the fitness tracker 150 and may inquire from the end user to confirm whether he or she is participating in exercising by selecting the appropriate button 1210, 1215 on the screen.

FIG. 13 provides an example of a message displayed on the screen 1300 of an end user device 140 informing the end user that he or she needs to wait one hour before taking a medication due to possible interactions. Here, the message in this example may have been provided to the end user based at least in part on an analysis conducted in response to the end user triggering a medication access request for the medication. In addition, an electronic user notification 1310 in the form of flashing light is being provided via a medication dispensing device 155 associated with a pill bottle holding the medication to caution the end user not to take the medication at that moment.

### CONCLUSION

Many modifications and other embodiments of the disclosure set forth herein will come to mind to one skilled in the art to which these modifications and other embodiments pertain having the benefit of the teachings presented in the foregoing descriptions and the associated drawings. Therefore, it is to be understood that the disclosure is not to be limited to the specific embodiments disclosed and that modifications and other embodiments are intended to be included within the scope of the appended claims. Although specific terms are employed herein, they are used in a generic and descriptive sense only and not for purposes of limitation.

1. A computer-implemented method for causing a medication dispensing device to generate one or more dynamic electronic user notifications, the computer-implemented method comprising:
receiving, by one or more processors of a user computing entity and originating from the medication dispensing device, an attempt data object, wherein the attempt data object indicates that an end user has communicated a medication access request for a medication to the medication dispensing device; and
responsive to receiving the attempt data object:
receiving, by the one or more processors and originating from a predictive data analysis server computing entity, a predictive polypharmacy data object for the end user, wherein: (i) the predictive polypharmacy data object is generated by the predictive data analysis computing entity via processing an end user medication profile for the end user, an end user factor profile for the end user, and a polypharmacy profile for the medication using a polypharmacy prediction machine learning model, and (ii) the predictive polypharmacy data object indicates an inferred prediction about polypharmacy compatibility of the polypharmacy profile and the end user factor profile;
determining, by the one or more processors and based at least in part on the predictive polypharmacy data object, a confirmation data object, wherein the confirmation data object indicates a recommended response to the attempt data object; and
transmitting the confirmation data object to the medical dispensing device, wherein the medical dispensing device is configured to generate the one or more dynamic electronic user notifications based at least in part on the confirmation data object and communicate the one or more dynamic electronic user notifications to the end user.

2. The computer-implemented method of claim 1, wherein the one or more dynamic electronic user notifications identify to the end user whether to take a dosage of the medication by causing illumination in a first color indicating to the end user an approval for taking the particular medication, a second color indicating to the end user a disapproval for taking the particular medication, and a third color indicating to the end user an undetermined state for taking the particular medication.

3. The computer-implemented method of claim 1, wherein the attempt data object is generated when the medication dispensing device is within a predefined distance from the end user computing entity.

4. The computer-implemented method of claim 1 further comprising:
detecting by the medication dispensing device a movement of the medication dispensing device; and
responsive to detecting the movement, sending the attempt data object to the user computing entity.

5. The computer-implemented method of claim 1 further comprising, in response to receiving the predictive polypharmacy data object, transmitting the confirmation data object to a virtual assistant entity in communication with the user computer entity, wherein the virtual assistant entity is configured to generate one or more assistant-originated electronic user notifications based at least in part on the confirmation data object and communicate the one or more assistant-originated electronic user notifications to the end user.

6. The computer-implemented method of claim 1, wherein the confirmation data object indicates a disapproval of the end user taking the medication and the method further comprises transmitting a locking data object to the medication dispensing device to cause the medication dispensing device to prevent the end user from accessing the medication.

7. The computer-implemented method of claim 1, wherein the medication dispensing device comprises a storage unit for storing the medication, a covering for the storage unit, a lock for the storage unit, or a tag attached to the storage unit.

8. The computer-implemented method of claim 1, wherein:
the end user factor profile comprises a set of relevant factors;
the set of relevant factors are identified based at least in part on a medication factors data object;
the medication factors data object is generated via processing an end user health profile for the end user using a medication factors machine learning model; and
the medication factors data object comprises a medication factor score for each factor of a plurality of factors, the medication factor score for a factor of the plurality of factors indicating a probability of a relevance of the factor in generating the predictive polypharmacy data object.

9. An apparatus for causing a medication dispensing device to generate one or more dynamic electronic user notifications, the apparatus comprising at least one processor and at least one memory including program code, the at least one memory and the program code configured to, with the processor, cause the apparatus to at least:
receive, originating from the medication dispensing device, an attempt data object, wherein the attempt data object indicates that an end user has communicated a medication access request for a medication to the medication dispensing device; and
responsive to receiving the attempt data object:
receive, originating from a predictive data analysis server computing entity, a predictive polypharmacy data object for the end user, wherein: (i) the predictive polypharmacy data object is generated by the predictive data analysis computing entity via processing an end user medication profile for the end user, an end user factor profile for the end user, and a polypharmacy profile for the medication using a polypharmacy prediction machine learning model, and (ii) the predictive polypharmacy data object indicates an inferred prediction about polypharmacy compatibility of the polypharmacy profile and the end user factor profile;
determine, based at least in part on the predictive polypharmacy data object, a confirmation data object, wherein the confirmation data object indicates a recommended response to the attempt data object; and
transmit the confirmation data object to the medical dispensing device, wherein the medical dispensing device is configured to generate the one or more dynamic electronic user notifications based at least in part on the confirmation data object and communicate the one or more dynamic electronic user notifications to the end user.

10. The apparatus of claim 9, wherein the one or more dynamic electronic user notifications identify to the end user whether to take a dosage of the medication by causing illumination in a first color indicating to the end user an approval for taking the medication, a second color indicating to the end user a disapproval for taking the medication, and a third color indicating to the end user an undetermined state for taking the medication.

11. The apparatus of claim 9, wherein the medication dispensing device detects a movement of the medication dispensing device and responsive to detecting the movement, sends the attempt data object to the apparatus.

12. The apparatus of claim 9, wherein the at least one memory and the program code are configured to, with the processor, cause the apparatus to at least, in response to receiving the predictive polypharmacy data object, transmit the confirmation data object to a virtual assistant entity to cause the virtual assistant entity to generate one or more assistant-originated electronic user notifications based at least in part on the confirmation data object and communicate the one or more assistant-originated electronic user notifications to the end user.

13. The apparatus of claim 9, wherein the confirmation data object indicates a disapproval of the end user taking the medication and the at least one memory and the program code are configured to, with the processor, cause the apparatus to at least transmit a locking data object to the medication dispensing device to cause the medication dispensing device to prevent the end user from accessing the medication.

14. The apparatus of claim 9, wherein the medication dispensing device comprises a storage unit for storing the medication, a covering for the storage unit, a lock for the storage unit, or a tag attached to the storage unit.

15. A non-transitory computer storage medium comprising instructions for causing a medication dispensing device to generate one or more dynamic electronic user notifications, the instructions being configured to cause one or more computer processors to at least perform operations configured to:
receive, originating from the medication dispensing device, an attempt data object, wherein the attempt data object indicates that an end user has communicated a medication access request for a medication to the medication dispensing device; and
responsive to receiving the attempt data object:
receive, originating from a predictive data analysis server computing entity, a predictive polypharmacy data object for the end user, wherein: (i) the predictive polypharmacy data object is generated by the predictive data analysis computing entity via processing an end user medication profile for the end user, an end user factor profile for the end user, and a polypharmacy profile for the medication using a polypharmacy prediction machine learning model, and (ii) the predictive polypharmacy data object indicates an inferred prediction about polypharmacy compatibility of the polypharmacy profile and the end user factor profile;
determine, based at least in part on the predictive polypharmacy data object, a confirmation data object, wherein the confirmation data object indicates a recommended response to the attempt data object; and
transmit the confirmation data object to the medical dispensing device, wherein the medical dispensing device is configured to generate the one or more dynamic electronic user notifications based at least in part on the confirmation data object and communicate the one or more dynamic electronic user notifications to the end user.

16. The computer program product of claim 15, wherein the one or more dynamic electronic user notifications identify to the end user whether to take a dosage of the medication by causing illumination in a first color indicating to the end user an approval for taking the medication, a second color indicating to the end user a disapproval for taking the medication, and a third color indicating to the end user an undetermined state for taking the medication.

17. The computer program product of claim 15, wherein the one or more computer processors receive the attempt data object as a result of the medication dispensing device being within a predefined distance from the one or more computer processors.

18. The computer program product of claim 15, wherein the medication dispensing device detects a movement of the medication dispensing device and responsive to detecting the movement, sends the attempt data object to the one or more computer processors.

19. The computer program product of claim 15, wherein the instructions are configured to cause the one or more computer processors to at least perform operations configured to, in response to receiving the predictive polypharmacy data object, transmit the confirmation data object to a virtual assistant entity to cause the virtual assistant entity to generate assistant-originated one or more electronic user notifications based at least in part on the confirmation data object and communicate the one or more assistant-originated electronic user notifications to the end user.

20. The computer program product of claim 15, wherein the confirmation data object indicates a disapproval of the end user taking the medication and the instructions are configured to cause the one or more computer processors to at least perform operations configured to transmit a locking data object to the medication dispensing device to cause the medication dispensing device to prevent the end user from accessing the medication.
